(19) United States
(12) Patent Application Publication
(10) Pub. No.: US 2005/0080021 A1
(43) Pub. Date: Apr. 14, 2005

(54) Nitric oxide donating derivatives of stilbenes, polyphenols and flavonoids for the treatment of cardiovascular disorders

(76) Inventors: Joseph Tucker, Calgary (CA); David McCaffrey, Calgary (CA); Norman C.W. Wong, Calgary (CA)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/762,796

(22) Filed: Jan. 22, 2004

Related U.S. Application Data
(63) Continuation-in-part of application No. 10/222,013, filed on Aug. 15, 2002.
(60) Provisional application No. 60/509,156, filed on Oct. 7, 2003.
(60) Provisional application No. 60/510,669, filed on Oct. 10, 2003.
(60) Provisional application No. 60/510,342, filed on Oct. 10, 2003.

(51) Int. Cl. A61K 31/7024; A61K 31/455; A61K 31/401

(52) U.S. Cl. 514/25; 514/355; 514/408; 514/517; 514/548; 514/129; 514/509; 536/116; 558/20; 558/194; 558/488; 548/530; 546/315

(57) ABSTRACT

Compounds and methods are provided for treating patients suffering from any of hypercholesterolemia, vascular oxidative stress and endothelial dysfunction.

### CROSS REFERENCES TO RELATED APPLICATIONS

This application is a continuation-in-part of U.S. pending patent application Ser. No. 10/222,013, filed Aug. 15, 2002 and claims benefit of U.S. Provisional Patent Application Ser. Nos. 60/509,156, filed Oct. 7, 2003; 60/510,669, filed Oct. 10, 2003; and 60/510,342, filed Oct. 10, 2003, which are incorporated herein by reference in their entirety.

### FIELD OF INVENTION

The present invention relates to the field of synthesis and administration of nitric oxide donor substituted stilbenes, polyphenols and flavonoids and derivatives thereof suitable for incorporation into foods, pharmaceuticals, nutraceuticals and the methods of treating individuals in need with the same.

### BACKGROUND OF INVENTION

Cardiovascular disease is a general term used to identify a group of disorders of the heart and blood vessels including hypertension, coronary heart disease, cerebrovascular disease, peripheral vascular disease, heart failure, rheumatic heart disease, congenital heart disease and cardiomyopathies. The leading cause of cardiovascular disease is atherosclerosis, the build up of lipid deposits on arterial walls. Elevated levels of cholesterol in the blood are highly correlated to the risk of developing atherosclerosis, and thus significant medical research has been devoted to the development of therapies that decrease blood cholesterol.

Atherosclerosis is associated with endothelial dysfunction, a disorder wherein normal function of the vasculature lining is impaired, which contributes to the pathogenesis of atherosclerosis, in addition to being a prominent risk factor for numerous other cardiovascular disorders such as angina, myocardial infarction and cerebrovascular disease. Hallmarks of endothelial dysfunction include increased oxidative vascular stress and vasoconstriction, as well as elevated levels of cholesterol in the blood, which all promote one another to accelerate the development of cardiovascular disease. In order to most successfully disrupt the development of disease, improved therapeutic strategies against the multiple causal risk factors of cardiovascular disease are needed.

Cholesterol Metabolism

Due to its insolubility, cholesterol is transported in the blood by complexes of lipid and protein termed lipoproteins. Low density lipoproteins (LDL) are believed to be responsible for the delivery of cholesterol from the liver to other tissues in the body, and have thus become popularly referred to as “bad cholesterol.” LDL particles are converted from intermediate density lipoproteins (IDL) which were themselves created by the removal of triglycerides from very low density lipoproteins (VLDL). VLDL are synthesized out of triglycerides and several apolipoproteins in the liver, where they are then secreted directly into the bloodstream.

High density lipoproteins (HDL) are thought to be the major carrier molecules that transport cholesterol from extrahepatic tissues to the liver where it is catabolized and then eliminated in a process termed reverse cholesterol transport (RCT), thereby earning HDL the moniker of the “good cholesterol.” In the elimination process that occurs in the liver, cholesterol is converted to bile acids and then excreted out of the body.

Current Treatments for Hyperlipidemias

Currently approved cholesterol lowering drugs provide therapeutic benefit by attacking the normal cholesterol metabolic pathways at a number of different points. Bile acid binding resins, such as cholestyramine, adsorb to bile acids and are excreted out of the body, resulting in an increased conversion of cholesterol to bile acids, consequently lowering blood cholesterol. Resins only lower serum cholesterol a maximum of 20%, cause gastrointestinal side effects and can not be given concomitantly with other medications as the resins will bind to and cause the excretion of such other drugs.

Niacin inhibits lipoprotein synthesis and decreases production of VLDL particles, which are needed to make LDL. When administered at the high concentrations necessary to increase HDL levels, serious side effects such as flushing occur.

Fibrates, such as clofibrate and fenofibrate, are believed to activate transcription factors belonging to the peroxisome proliferator-activated receptor (PPAR) family of nuclear hormone receptors. These transcription factors up-regulate genes involved in the production of HDL and down-regulate genes involved in the production of LDL. Fibrates are used to treat hyperlipidemias because they reduce serum triglycerides by lowering the VLDL fraction. However, they have not been approved in the United States as hypercholesterolemia therapeutics, due to the heterogeneous nature of the lipid response in patients, and the lack of efficacy observed in patients with established coronary heart disease. As well, the use of fibrates is associated with serious side effects, such as gastrointestinal cancer, gallbladder disease and an increased incidence in non-coronary mortality.

Statins, also known as HMG CoA reductase inhibitors, decrease VLDL, LDL and IDL cholesterol by blocking the rate-limiting enzyme in hepatic cholesterol synthesis. Statins increase HDL levels only marginally, and numerous liver and kidney dysfunction side effects have been associated with the use of these drugs.

Ezetimibe is the first approved drug in a new class of cardiovascular therapeutics, which functions by inhibiting cholesterol uptake in the intestine. Ezetimibe lowers LDL but does not appreciably increase HDL levels, and does not address the cholesterol which is synthesized in the body nor the cholesterol circulating in the bloodstream or present in atherosclerotic plaques. Other compounds that have also been discovered to affect cholesterol absorption include the bile-acid binding agent cholestyramine and the phytosterols.

Despite the development of these therapeutic approaches, little has been achieved to increase the blood levels of HDL, and all of the drugs currently approved are limited in their therapeutic effectiveness by side effects and efficacy. Consequently, there is a need for improved therapeutic approaches to safely elevate HDL and thus increase the rate of reverse cholesterol transport to reduce blood levels of cholesterol.

Endothelial Dysfunction and Atherosclerosis

Impaired endothelial function occurs early in the genesis of atherosclerosis, and in fact is detectable before lipid deposits. Endothelial dysfunction is symptomatically characterized by vasoconstriction and leads to hypertension, which is a well known risk factor for other cardiovascular disorders such as stroke and myocardial infarction. Research has causally linked the diminished endothelial function in atherosclerosis patients to reduced bioavailability of nitric oxide (NO), a signaling molecule that induces vasodilation.

Decreased bioavailability of NO also activates other mechanisms that play a role in the pathogenesis of atherosclerosis. For instance NO is well known to inhibit platelet aggregation, a necessary step in the development of the lipid plaques that characterize atherosclerosis. As well, NO is an important endogenous mediator that inhibits leukocyte adhesion, which is a major step in the development of atherosclerosis and is probably the result of increased vascular oxidative stress in hyperlipidemic patients. Adherent leukocytes further increase oxidant stress by releasing large amounts of reactive oxygen species.

Increased vascular oxidative stress and hypercholesterolemia have individually been identified as contributors to the cause of reduced NO bioavailability. Increased oxidation also leads to free-radical mediated lipid peroxidation, another inducer of atherosclerotic lesion formation. In summary, it would appear that a positive feedback loop exists wherein each of these three major factors, hypercholesterolemia, vascular oxidative stress and reduced bioavailability of NO, increases the extent and pathological severity of the other factors.

Nitric Oxide as a Therapy for Atherosclerosis-Endothelial Dysfunction

Therapeutic modalities that employ compounds known to donate NO have been employed clinically in an attempt to break the atherosclerosis-endothelial dysfunction cycle without success. Exogenous NO released by NO-donating drugs has been demonstrated to generate not only NO but also peroxynitrite anion, a potent oxidant, which further increases oxidative stress. The generation of peroxynitrite by NO-donors and subsequent down regulation of responsiveness to NO caused by increased oxidative stress may underlie the well documented tolerance that develops in patients treated chronically with organic nitrate esters. NO-donating drugs are also believed to require a transition through a thiol intermediate prior to their liberation of NO, and the bioavailability of thiols is significantly diminished in such conditions of oxidative stress.

Anti-Oxidant/Nitric Oxide Combined Therapy

In an attempt to mitigate the exacerbation to vascular oxidative stress caused by NO-donating drugs, anti-oxidants have been provided to patients in combination with NO-donors. Some studies have demonstrated that combination of anti-oxidants with NO-donors significantly increased endothelial-dependent vasodilation in hypercholesteremic subjects.

However, these results are challenged by others who have not found improved endothelium-dependent vasodilation with this therapeutic approach, possibly due to difficulties in achieving sufficient intracellular dosage, and by the fact that NO-donor treatment has thus far not been correlated to any delay of the development of atherosclerosis or an increase in the life expectancy of patients with active atherosclerosis. Additionally, neither NO-donor nor NO-donor/anti-oxidant combined therapies addresses directly the hypercholesterolemia facet of the atherosclerosis-endothelial dysfunction cycle.

The combination of NO-donating and anti-oxidant agents with existing therapies that treat the hypercholesterolemia underlying atherosclerosis is also a suboptimal approach, as the currently approved drugs do not effectively exploit the use of increasing HDL to efficiently transport cholesterol out of the body.

However, one might hypothesize that a preferred anti-oxidant/NO-donor combination would ensure that the anti-oxidant and NO-donor were present in the same location and same time in the body, in order for the anti-oxidant to most effectively counteract the potential oxidative side effects of NO. The difficulty in meeting this need using a combination of several different drugs with differing release rates and bioavailability is likely to be exacerbated by the short half life of NO in the cellular environment once released from the donor molecule.

Stilbenes, Polyphenols and Flavonoids as Anti-Oxidants

Reactive oxygen species (ROS), which can be produced by normal cellular respiration, are a major cause of oxidative damage in the body. One of the most effective methods to counter ROS is to “mop” up the reactive groups by providing an anti-oxidant compound which binds to the ROS and thus prevents them from inappropriately bonding to key proteins and DNA in the cell. Very effective anti-oxidant compounds capable of eliminating ROS often contain at least one phenolic ring structure. A phenol ring is a reactive species with which a ROS may form a covalent bond, which thereby abolishes the strong oxidative reactivity of the ROS. Stilbenes, polyphenols and flavonoids all contain at least two phenolic ring structures, thereby making them potentially effective as anti-oxidant agents.

Resveratrol, Other Stilbenes and Polyphenols, and Flavonoids as Pro-Apolipoprotein A1 Agents

In addition to their anti-oxidant activities stilbenes, polyphenols and flavonoids also have activities useful for the treatment of hypercholesterolemia. For example, one well known stilbene, resveratrol (3,4′,5-trihydroxy trans stilbene), a naturally occurring polyphenol found in certain plants, has been suggested to underlie the epidemiological observation termed the “French Paradox.” This paradox refers to the observation that the French population suffers from one third the incidence of cardiovascular disease of the North American population despite the comparable high fat diet. The French Paradox has been correlated to the high quantities of red wine consumed by the French population compared to that consumed by the North American population. Resveratrol is highly abundant in the skin of red grapes and thus is found in significant quantities in red wine but is almost completely absent in white wine or other alcoholic beverages.

The mechanism by which resveratrol reduces the incidence of cardiovascular disease remains a topic of considerable debate, with several competing hypotheses. Resveratrol has been demonstrated to be a potent anti-oxidant, which is suggested to result in lower levels of peroxidation of LDL particles, and subsequently to inhibit atherogenesis. Resveratrol has also been implicated as an inhibitor of leukocyte adhesion and platelet aggregation. In addition, resveratrol is being investigated as a potential anti-cancer therapeutic due to its described capability of modulating the activity levels of p21 and p53.

Resveratrol has been identified as an anti-inflammatory agent, with proposed mechanisms including the inhibition of the cyclooxygenase-1 enzyme (See U.S. Pat. No. 6,541,045; Jayatilake et al. J Nat Prod. 1993 October; 56(10):1805-10; U.S. Pat. No. 6,414,037) and protein kinase inhibition (U.S. patent application 0030171429). Consequently, resveratrol may have the potential to be employed therapeutically to treat arthritic disorders, asthmatic disorders, psoriatic disorders, gastrointestinal disorders, ophthalmic disorders, pulmonary inflammatory disorders, cancer, as an analgesic, as an anti-pyretic, or for the treatment of inflammation that is associated with vascular diseases, central nervous system disorders and bacterial, fungal and viral infections.

Resveratrol was recently described as a sirtuin-activating compound, and was suggested to increase longevity through a direct interaction with SirT1, leading to down-regulation of p53. Resveratrol is also known to antagonize the aryl hydrocarbon receptor and agonize the estrogen receptor, and has been described to mediate activity through activation of the ERK 1/2 pathway and through increasing the activity of the transcription factor egr-1.

More recently, we have shown that resveratrol has the ability to increase the transcription of apolipoprotein A1, putatively mediated through Site S, a nucleotide sequence in the promoter region of the ApoA-1 gene (PCT/CA03/01220).

A sequence, AGCCCCCGC, found within Site S, has been described as an “Egr-1 response element” consensus sequence. This motif is contained within the nucleotides spanning −196 to −174 of the human APO I promoter (Kilbourne et al. 1995, JBC, 270(12):7004-10). Without being bound by any particular theory, this AGCCCCCGC element found to be contained within Site S is a sequence through which resveratrol is proposed to mediate its activity, but this is not to the exclusion of other potentially required elements.

It is believed that a nucleotide sequence comprising Site S or about any 8 contiguous bases of the AGCCCCCGC element act as an enhancer element when operably linked to a heterologous promoter in order to modulate the expression of a reporter gene.

While not being limited to a theory, resveratrol is believed to cause the previously described effects due to its molecular structure, the reactive and necessary core consisting of at least one aromatic ring structure, with at least one hydroxyl group located on an aromatic ring. Naturally produced resveratrol itself is specifically comprised of two aromatic rings, with two hydroxyls located at the 3 and 5 positions on one ring and one hydroxyl located at the 4′ position on the other, and the two aromatic rings are connected by two carbon atoms which have a double bond between them. Other compounds of this general class, said class being those compounds which comprise at least one aromatic ring structure with at least one hydroxyl group located on the ring, are believed to possess the same capabilities and to produce the same results as those listed for resveratrol.

Consequently, stilbenes, which comprise two aromatic rings linked by two carbon atoms, other polyphenols, such as those comprising two or more aromatic rings, preferably two, linked by one, two or three atoms, said atoms independently selected from the group consisting of nitrogen, carbon, oxygen and sulfur, and which may or may not be independently substituted with side groups such as ketone oxygens, and flavonoids, such as but not limited to naturally occurring flavonoids, such as but not limited to naringenin, quercetin, piceatannol, butein, fisetin, isoliquiritigenin, and hesperitin, are all compounds possess similar properties as those described for resveratrol. As a result, it has been discovered that any of these compounds may be considered to be functionally interchangeable with resveratrol when utilized for the prevention or treatment of diseases, disorders or conditions, especially but not limited to those diseases, disorders or conditions associated with cholesterol, cardiovascular disease, hypertension, oxidative damage, dyslipidemia, apolipoprotein A1 or apoB regulation, or in modifying or regulating other facets of cholesterol metabolism such as inhibiting HMG CoA reductase, increasing PPAR activity, inhibiting ACAT, increasing ABCA-1 activity, increasing HDL, or decreasing LDL or triglycerides. Flavonoids that do not have nitric oxide donating moieties attached have previously been taught as having potential serum cholesterol reducing activities, for example in U.S. Pat. Nos. 5,877,208, 6,455,577, 5,763,414, 5,792,461, 6,165,984, and 6,133,241.

Similarly, any of the stilbenes, other polyphenols and flavonoids of this class may be considered to be functionally interchangeable with resveratrol when utilized to modulate transcription from site S, from the AGCCCCCGC element, or when utilized to inhibit leukocyte adhesion or platelet aggregation, or to inhibit COX-1. This is not to imply that all of the compounds will be identical in terms of the level of activity for each of these functions or capabilities, or for in vivo toxicity or efficacy, or for bioavailability. These compounds demonstrate, over the course of simple testing, easily performed by one of skill in the art and not requiring undue experimentation, that some provide improved capabilities or functionality relative to others, and are therefore preferred over others as therapeutic agents.

Drawbacks to the Therapeutic Use of Stilbenes, Flavonoids and Other Polyphenols

Unfortunately, the use of stilbenes, such as resveratrol, and other polyphenols, and flavonoids as therapeutic agents can be problematic.

The most abundant and available source of resveratrol for consumers, red wine, can not be consumed in substantial quantities on a daily basis due to the numerous well documented deleterious effects of excessive alcohol consumption. That is to say, the actions of resveratrol may be better or safer in the absence of alcohol.

Many stilbenes, polyphenols and flavonoids with potential beneficial qualities may be created that are not naturally synthesized and have not yet been described or made available for testing. Such compounds must be created in the laboratory and tested in appropriate in vitro and in vivo assays to demonstrate beneficial therapeutic activities before being examined in human clinical studies.

Numerous stilbenes, polyphenols and flavonoids of biological origin are known, as they are often synthesized by plants. Many of these compounds have been examined for potentially beneficial properties, such as their known in vitro anti-oxidant capabilities, their putative anti-cancer efficacy and their apparent beneficial effects on cardiovascular disease. While several human studies have been conducted on such compounds, the results have been thus far unclear and occasionally contradictory. For example, the findings of human clinical studies have yet to demonstrate unequivocal evidence of benefit on primary clinical endpoints such as atherosclerotic plaque size, or reduction in cardiovascular events such as heart attacks. In some cases findings from animal studies, using for example rabbits or rats, have not correlated with the results of human studies. For example, whereas administration of naringenin (as one example flavonoid of many components found in administered citrus juice) was observed to increase HDL but have no effect on LDL or triglycerides in a human study, when administered to rabbits naringenin was found to decrease LDL but have no effect upon HDL.

Additionally, no clinical studies to date have described the appropriate dosage of flavonoids such as naringenin, or stilbenes such as resveratrol, or other polyphenols to use for human therapy in the treatment of cardiovascular disorders.

Compounds with Protecting Groups may Experience Longer Serum Half Lives, Improved Efficacy, Reduced Toxicity and Improved Therapeutic Outcome

Compounds administered as therapeutic agents to individuals in need are typically metabolized in the body to a variety of metabolites prior to excretion. Such metabolites often differ from the parent compound in terms of toxicity, efficacy and length of residence in the serum. For many compounds, the metabolites are not as effective as the parent compound and can be more toxic.

In the metabolism of exogenously administered therapeutic compounds, a number of different modifications may occur, for example the addition of various chemical moieties or removal of key groups. One metabolic reaction that occurs in vivo is the removal of hydroxyl groups. The removal of hydroxyl groups from compounds with a core structure of flavonoids, stilbenes, and other polyphenolic compounds, the nitric oxide donating derivatives of which comprise compounds of the invention, may significantly reduce the ability of the compounds to positively affect cholesterol metabolism, as the aforementioned hydroxyl groups are an integral part of the active site of such molecules. It therefore advantageously improves the beneficial effect of administration of compounds of the invention if some mechanism is utilized for the protection of the hydroxyl groups to reduce the rate of metabolism and thus increase the time for which the compounds remain in the body.

One protection mechanism commonly employed in laboratory chemical reactions is the use of protecting groups, which are attached to an easily modified, labile chemical group of a larger molecule, in order to prevent the modification or loss of the labile group. Protecting groups may be later removed to restore the original molecule, with no changes to any of the covalent bonds in the entire molecule. A similar form of protection may be used for compounds intended to be administered to a patient, where known reactions in the body are likely to occur that will reconstitute the active molecule. The speed at which protecting groups are released from a molecule may be controlled to affect the rate at which the drug is released. As well, it is known that phenolic hydroxyl groups, such as those found in compounds contemplated by the present invention, are prone to glucoronidation and sulfation reactions that facilitate excretion. Protection against these reactions by blocking the phenolic hydroxyl group with another chemical group, such as a nitric ester (also referred to as an organic nitrate or ONO.sub.2) group, further extends a molecule's half life in the body and postpones excretion.

As an example, resveratrol, which contains three putatively important and therapeutically active hydroxyl groups, may be protected by the replacement of the hydroxyl groups with nitric esters (also known as nitrates, nitrooxy groups, or ONO.sub.2 and are occasionally referred to as nitroxy, but which should not be confused with NO.sub.2) which are replaced over time while in the body with hydroxyl groups to reconstitute the active compound, resveratrol. As the nitric esters are replaced with hydroxyl groups one at a time over a period, and the resveratrol molecule comprising one or two nitric esters is still partially active, the effective half life in the body of resveratrol activity is increased. Such a strategy further permits the use of lower doses of the nitrate form of resveratrol relative to the parent, hydroxylated form of resveratrol, which then results in lower side effects in the patient. Obviously, such an approach would also be effective for the other stilbenes, other polyphenols, and flavonoids contemplated in the invention as they also are contemplated to comprise one or more hydroxyl groups that may form an integral part of the molecule's active site.

A Need Exists for Improved Cardiovasulcar Therapy

In view of the foregoing, it is evident that there is a need for the development of improved therapies and compounds that can safely and effectively lower blood cholesterol while simultaneously decreasing endothelial dysfunction and decreasing vascular oxidative stress.

### SUMMARY OF INVENTION

It is an aspect of this invention to provide classes of novel compounds that have the ability to donate nitric oxide concomitant and co-localized with the release of a free radical scavenging anti-oxidant molecule, and methods of treatment therewith. These novel compounds simultaneously have the capability to induce the expression of ApoA1 and thereby increase the blood levels of HDL and lower blood levels of cholesterol. In addition these compounds have other beneficial activities, including those of inhibiting HMG-CoA reductase, increasing PPAR activity, inhibiting ACAT, increasing ABCA-1 activity, and decreasing blood levels of LDL and triglycerides. The combined multivariate effects of these compounds may be used to decrease endothelial dysfunction, decrease vascular oxidative stress and decrease hyperlipidemia, and thereby treat cardiovascular disorders such as atherosclerosis, hypertension, coronary artery disease, cerebrovascular disease and the like.

It is a another aspect of the present invention to provide classes of novel compounds that may be used to increase transcription factor binding to egr-1 like promoter sequences, thereby modulating the expression of cancer related genes such as p21 and p53 for treating cancer, and to provide new methods of treatment therewith.

It is a further aspect of the present invention to provide classes of novel compounds that may be used to increase transcription factor binding to egr-1 like promoter sequences, thereby modulating the expression of longevity related genes such as the sirtuins, and to thereby extend the life-span of an individual so treated, and to provide new methods of treatment therewith.

In accordance with the various aspects and principles of the current invention there are provided compounds in accordance with the following.

A stilbene compound comprising the following structure: 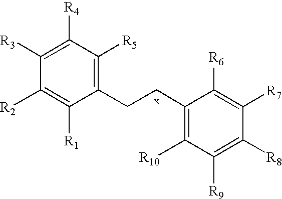
wherein
R1, R2, R3, R4, R5, R6, R7, R8, R9 and R10 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [OCOR11], O-sulfate [the sulfate conjugate], or O-glucoronidate [the glucoronic (AKA glucuronic) acid conjugates], with the proviso that at least one of R1-R10 is nitrooxy; and
wherein OCOR11 means 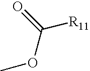
   wherein R11 can be C₁₋₁₈, aryl, heteroaryl, and optionally substituted derivatives thereof; and
wherein X can be a single, double or triple bond.
A flavonoid compound comprising the following structure: 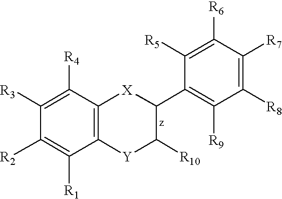
   wherein
R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [OCOR11], O-sulfate [the sulfate conjugate], or O-glucoronidate [the glucoronic (AKA glucuronic) acid conjugates], with the proviso that at least one of R1-R10 or R12 or R13 is nitrooxy;
OCOR11 means 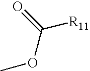
   wherein R11 can be C₁₋₁₈, aryl, heteroaryl, and optionally substituted derivatives thereof;
X can be O or CR12;
Y can be CO [a ketone still maintaining the 6 atom ring structure] or CR13; and Z can be a single or a double bond.
An isoflavonoid compound comprising the following structure: 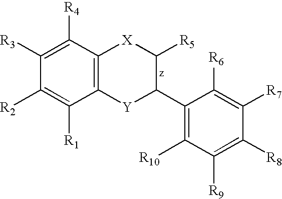
   wherein
R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 or R12 or R13 is nitrooxy; OCOR11 means 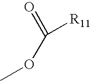
   wherein R11 can be C₁₋₁₈, aryl, heteroaryl, and optionally substituted derivatives thereof;
X can be O or CR12;
Y can be CO [a ketone still maintaining the 6 atom ring structure] or CR13; and Z can be a single or a double bond.

A chalcone compound comprising the following structure: 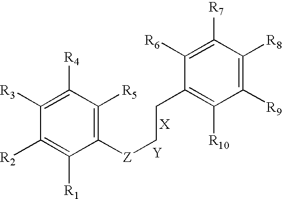
wherein

R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 or R12 or R13 is nitrooxy;
OCOR11 means 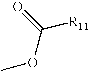
   wherein R11 can be C₁₋₁₈, aryl, heteroaryl, and optionally substituted derivatives thereof;
X can be a single or a double bond;
Y can be a single or a double bond; and
Z can be CO [a ketone] or CR13;
with the proviso that X and Y are not both double bonds, and if Z is CO then Y is not a double bond.
A polyphenol compound comprisng the following structure: 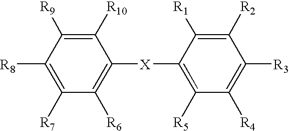
   wherein
R1, R2, R3, R4, R5, R6, R7, R8, R9 and R10 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 is nitrooxy;
OCOR11 means 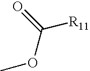
   wherein R11 can be C₁₋₁₈, aryl, heteroaryl, and optionally substituted derivatives thereof;
X can be C, S, (CO), SO, AKA ketone, (SO.sub.2)N, (CO)C, (CO)N, (CO)O, C—N [single bond], C═N [double bond], C—O, N—O, N—N [single bond], or N═N [double bond].

In addition, there are provided methods for treating cardiovascular, cholesterol or lipid related disorders in a patient comprising administering to a patient in need of treatment a therapeutically effective amount of any of the foregoing compounds. Another preferred treatment method for inducing expression of ApoA1 while providing anti-oxidant activity in a patient comprises administering to said patient any of the foregoing compounds. Still other preferred methods of the present invention for reducing serum cholesterol in a patient comprise administering to said patient any of the foregoing compounds.

### DETAILED DESCRIPTION OF THE INVENTION AND BEST MODE

In accordance with the principles and aspects of the present invention, methods and compounds are provided for treating cardiovascular disorders along with descriptions characterizing the potential mechanisms of action in detail regarding the use of NO-donating analogues of stilbenes, polyphenols and flavonoids and derivatives thereof. Understanding the potential mechanisms of action may lead to the improved development of still more derivatives and analogues with further enhanced therapeutic benefit, that are also within the scope of the present invention.

It is clear that there are many factors influencing the pathogenesis of cardiovascular disorders. It is also evident that an approach which simultaneously treats the three major facets of developing disease, namely increasing vascular oxidative stress, reducing bioavailability of nitric oxide and of hypercholesterolemia, is lacking in modern medicine. Consequently, this invention details a methodology that addresses all three factors simultaneously by providing in a single novel molecule anti-oxidant activity, nitric oxide releasing activity, and the capability to induce reverse cholesterol transport. The compounds and methods of treatment of the invention are made still more efficacious by the fact that the anti-oxidant capability and nitric oxide donation occur simultaneously upon the release of nitric oxide and are therefore particularly preferred. It is readily apparent to one of skill in the art that NO-donating moieties of any type may be advantageously attached to almost any portion of the stilbenes, other polyphenols and flavonoids which form the core of the compounds contemplated by the present invention, or to any derivative of such compounds that retains the anti-oxidant property and induction of apolipoprotein A1 transcription capability or otherwise has the activity of increasing reverse cholesterol transport or of reducing serum cholesterol or indeed to any compound that comprises both anti-oxidant and serum cholesterol-decreasing capabilities, and still retain the activities which are provided for in this invention.

Compounds provided by the present invention include analogues of resveratrol, other stilbenes, other polyphenols, and flavonoids, with attached moieties that are capable of releasing nitric oxide when administered to a patient. Such compounds include but are not limited to analogues of resveratrol, other stilbenes, other polyphenols, and flavonoids, wherein the nitric oxide donating moieties belong to the organic nitrate, alkoxynitrate, diazeniumdiolate, thionitroxy, and the like classes of chemical structures.

The present invention also provides for the synthesis, composition and methods of treatment for nitrooxy derivatives of compounds other than the above described stilbenes, polyphenols and flavonoids, wherein the said compounds from which the nitrooxy derivatives are synthesized contain aromatic or heteroaromatic rings, one or more hydroxyl groups, and are known to modulate serum cholesterol levels. One examplar class of compounds that contain aromatic or heteroaromatic rings, one or more hydroxyl groups, and are known to modulate serum cholesterol levels comprise HMG CoA reductase inhibitors, also known as statins. Commercially available statins, the nitrooxy derivatives of which are provided for in this invention, comprise atorvastatin, lovastatin, pravastatin, simvastatin, fluvastatin, cerivastatin, and rosuvastatin. Two other compounds that fall within the specification of containing aromatic or heteroaromatic rings, one or more hydroxyl groups, and known to modulate serum cholesterol levels are ezetimibe and niacin. The nitrooxy derivatives of ezetimibe and niacin are therefore also provided for in this invention.

Synthesis of Nitric Oxide Donating Derivatives of Stilbenes, Polyphenols, Flavonoids, Statins and Ezetimibe

Organic nitrate (also referred to as nitrooxy, nitric esters, ONO.sub.2 and occasionally as “nitroxy” but which is not to be confused with NO.sub.2) groups may be added to compounds using known methods, such as that of Hakimelahi wherein the nitrooxy group is substituted for existing hydroxyl groups on the parent molecule (Hakimelahi et al. 1984. Helv. Chim. Acta. 67:906-915).

Alkoxynitroxy groups may be added to compounds using, for example, the methods taught in U.S. Pat. No. 5,861,246. Diazeniumdolates may be synthesized by various methods including, for example, the methods taught in U.S. Pat. Nos. 4,954,526, 5,039,705, 5,155,137, 5,405,919 and 6,232,336, all of which are fully incorporated herein by reference.

Nitric oxide donating moieties may be advantageously attached to a stilbene, such as resveratrol, a polyphenol, or a flavonoid, such as naringenin, or other compounds as described and provided for in this invention, such as a member of the class of statins, or a derivative or analogue thereof via a covalent or ionic bond. Preferably, the nitric oxide donating moiety or moieties are attached by one or more covalent bonds. Nitric oxide donating moieties may be advantageously attached to any portion of the molecule. In one embodiment, nitric oxide donating moieties are substituted in place of one or more hydroxyl groups. In a preferred embodiment, the substitutions are of organic nitrate groups in place of hydroxyl groups. In another preferred embodiment, the nitric oxide donating moieties have replaced all of the hydroxyl groups of the stilbene, such as resveratrol, the polyphenol, or the flavonoid, such as naringenin, or other compounds as described and provided for in this invention, such as any member of the class of statins, or those hydroxyl groups of an analogue or derivative thereof.

For all of the compounds of the invention, substitution of a hydroxyl group with a fluoride ion is also contemplated and provided for, as such modifications to parent compounds are commonplace, known to increase drug stability without altering the mechanism of action, and are readily accomplished by one of skill in the art.

For all of the compounds of the invention, acetylated-derivatives of the compounds are also contemplated and provided for, as such modifications to parent compounds are commonplace, known to improve the beneficial effects of the drug without altering the mechanism of action, and are readily accomplished by one of skill in the art.

For all of the compounds of the invention, phosphorylated-derivatives of the compounds are also contemplated and provided for, as such modifications to parent compounds are commonplace, known to improve the beneficial effects of the drug without altering the mechanism of action, and are readily accomplished by one of skill in the art.

Glucoronidated derivatives of the compounds contemplated by the invention are also contemplated herein, as glucoronidation is a process that naturally occurs in the body as part of the metabolism of stilbenes, other polyphenols, and flavonoids. Once provided to a patient, much of the compounds of the invention will be modified in the body and will therefore be present in the body in glucoronidated form. The conjugation of glucoronic acid to the compounds of the invention prior to administration will therefore not preclude the function or therapeutic utility of the compounds as determined by in vivo studies. As a result, compounds of the invention with an additional sugar moiety attached are considered to be functionally comparable to the parent compounds, and are therefore provided for in the present invention. Glucoronidation of any stilbene, polyphenol or flavonoid derivative compound contemplated by the present invention may be achieved, for example, using human liver microsomes as in the method of Otake (Otake et al. 2002. Drug Metab. Disp. 30(5):576-581).

Similarly, sulfated derivatives of the compounds contemplated by the invention are also contemplated herein, as sulfation is a process that naturally occurs in the body as part of the metabolism of stilbenes, other polyphenols, and flavonoids. Once provided to a patient, some of the compounds of the invention will be modified in the body and will therefore be present in the body in sulfated form. Sulfation will therefore not preclude the function or therapeutic utility of the compounds as determined by in vivo studies. As a result, compounds of the invention that have been subjected to a sulfation reaction are considered to be functionally comparable to the parent compounds, and are therefore provided for in the present invention. Sulfation of any stilbene, polyphenol or flavonoid derivative compound contemplated by the present invention may be achieved, for example, using the ion-air extraction method of Varin (Varin et al. 1987. Anal. Biochem. 161:176-180).

Salts of the compounds described herein, including those preferred for pharmaceutical formulations, are also provided for in this invention.

Compounds Contemplated by the Invention

In order to clarify the compounds provided for in the present invention we here present illustrative chemical structures, but this is not to limit the scope of the invention to the compounds listed below. When the term “nitrooxy” is used, what is meant is the nitric ester group —ONO₂. When the terms “hydroxyl” or “hydroxy” are used, what is meant is the group —OH.

The present invention provides for compounds having the general stilbene structure: 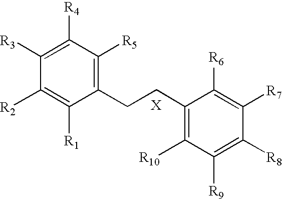
which can be further subdivided into the following structures: 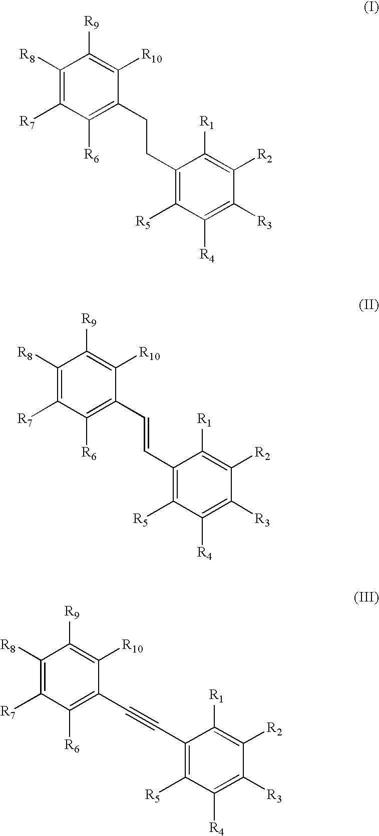
wherein
R1, R2, R3, R4, R5, R6, R7, R8, R9 and R10 may each be independently hydrogen, hydroxyl, nitrooxy, fluoride, methoxy, ethoxy, phosphate, acetyl, O-sulfate or O-glucoronidate, with the proviso that at least one of R1-R10 is nitrooxy

The present invention also provides for compounds of the following general structures: 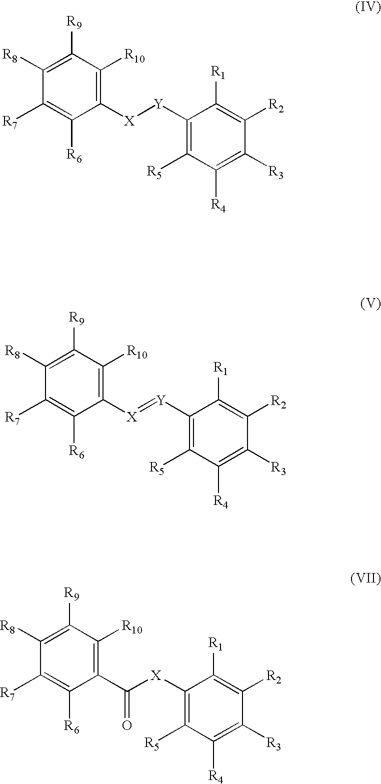
Wherein
R1, R2, R3, R4, R5, R6, R7, R8, R9 and R10 may each be independently hydrogen, hydroxyl, nitrooxy, fluoride, O-sulfate or O-glucoronidate.
X and Y may each independently be C, N, O, with the proviso that if either of X or Y is C then the other is not C.

The present invention also provides for compounds of the following general structure: 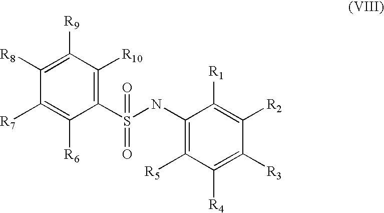
Wherein
R1, R2, R3, R4, R5, R6, R7, R8, R9 and R10 may each be independently hydrogen, hydroxyl, nitrooxy, fluoride, O-sulfate or O-glucoronidate.

The present invention also provides for compounds having the general polyphenol structure: 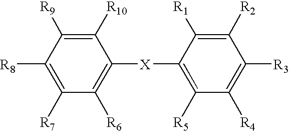
which can be further subdivided into the following structures: 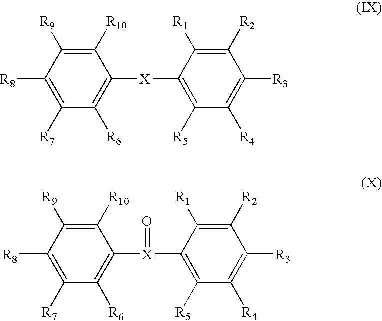
Wherein
X is C or S
R1, R2, R3, R4, R5, R6, R7, R8, R9 and R10 may each be independently hydrogen, hydroxyl, nitrooxy, fluoride, O-sulfate or O-glucoronidate.

The present invention also provides for compounds having the general flavonoid structure: 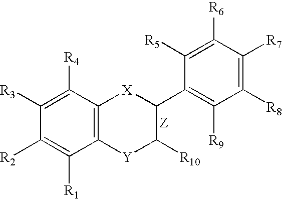
which can be further subdivided into the following structures:

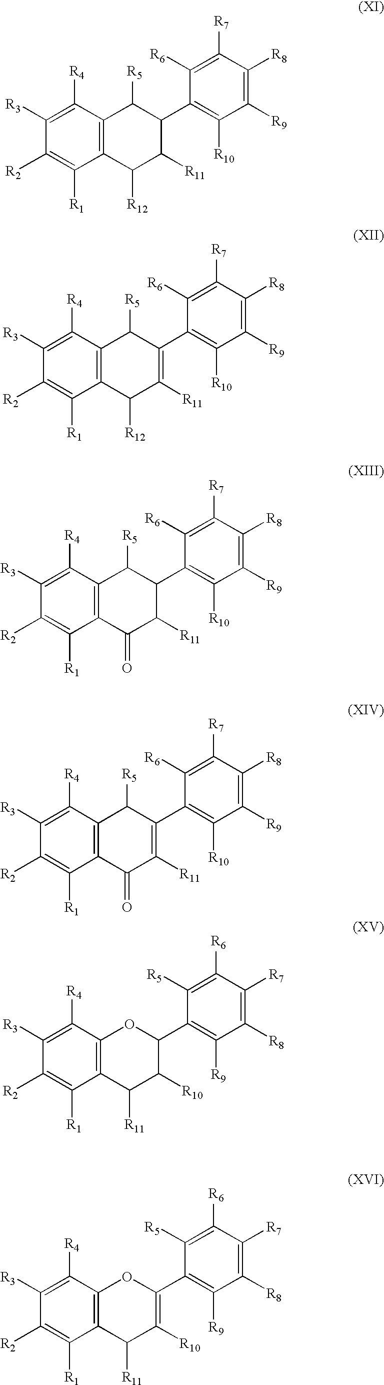

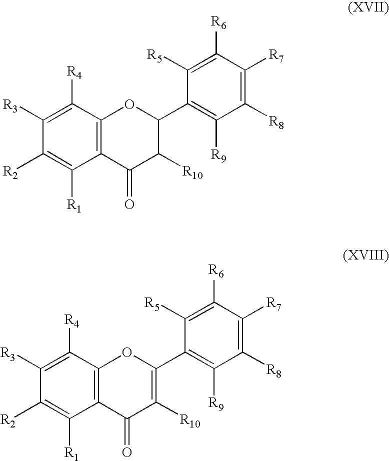

The present invention also provides for compounds having the general isoflavonoid structure: 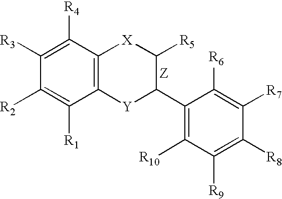
which can be further subdivided into the following structures:

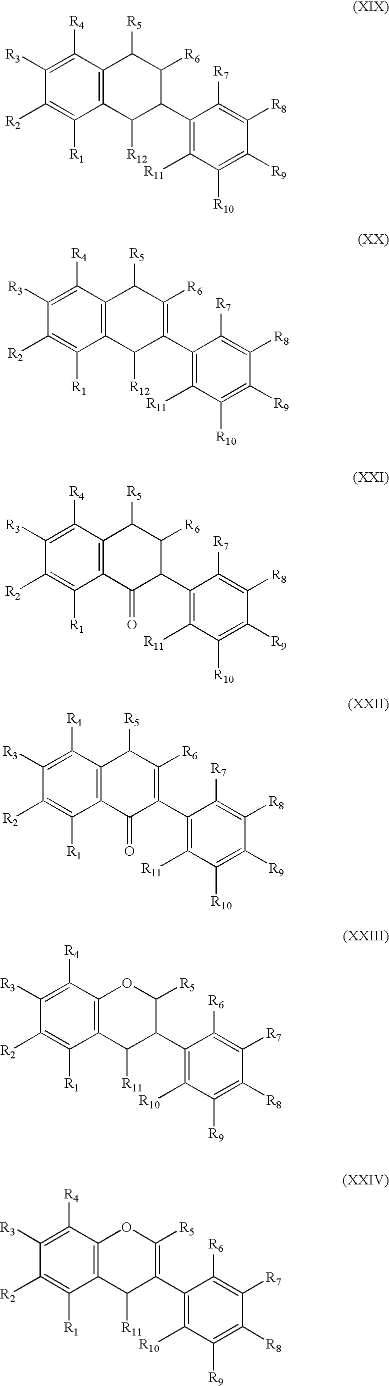

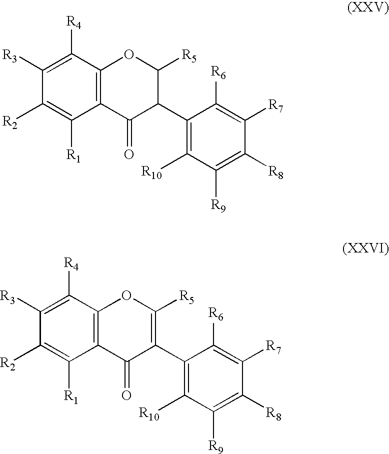

The present invention also provides for compounds having the general chalcone structure: 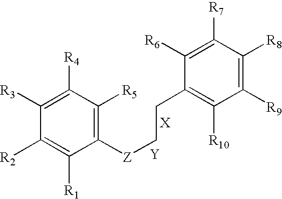
which can be further subdivided into the following structures: 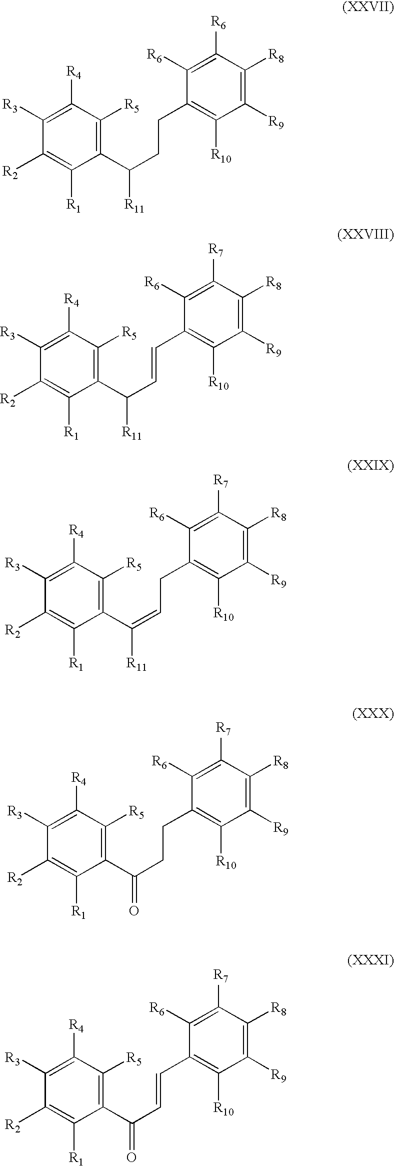
wherein
R1, R2, R3, R4, R5, R6, R7, R8, R9, R10, R11 and R12 may each be independently hydrogen, hydroxyl, nitrooxy, fluoride, O-sulfate or O-glucoronidate.

Other NO-donating derivatives of cholesterol lowering compounds provided for in the invention include:

The present invention also provides for compounds of the following general formula 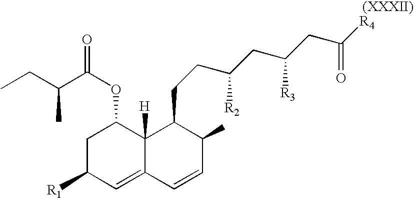
Wherein R1, R2, R3 and R4 are each independently hydroxyl or nitrooxy, provided that at least one of R1, R2, R3 and R4 is nitrooxy.

The present invention also provides for the compound 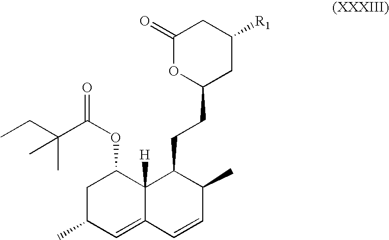
Wherein R1 is nitrooxy.

The present invention also provides for the compound 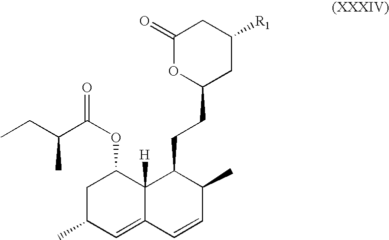
Wherein R1 is nitrooxy.

The present invention also provides for compounds of the following general formulae 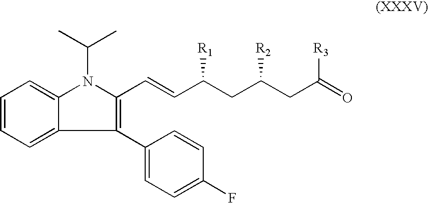
Wherein R1, R2 and R3 are each independently hydroxyl or nitrooxy, provided that at least one of R1, R2 and R3 is nitrooxy.

The present invention also provides for compounds of the following general formulae 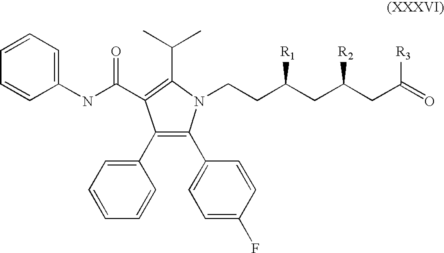
Wherein R1, R2 and R3 are each independently hydroxyl or nitrooxy, provided that at least one of R1, R2 and R3 is nitrooxy.

The present invention also provides for compounds of the following general formulae 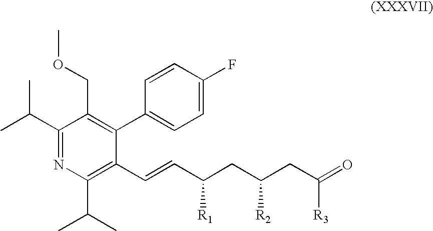
Wherein R1, R2 and R3 are each independently hydroxyl or nitrooxy, provided that at least one of R1, R2 and R3 is nitrooxy.

The present invention also provides for compounds of the following general formulae 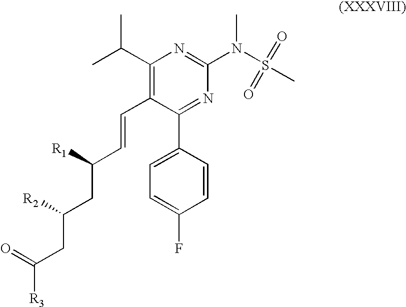
Wherein R1, R2 and R3 are each independently hydroxyl or nitrooxy, provided that at least one of R1, R2 and R3 is nitrooxy.

The present invention also provides for compounds of the following general formula 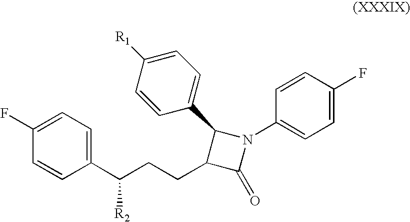
Wherein R1 and R2 are each independently hydroxyl or nitrooxy, provided that at least one of R1 and R2 is nitrooxy.

The present invention also provides for the compound 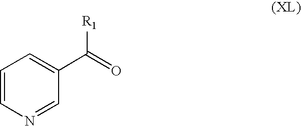
Wherein R1 is nitrooxy.
Methods for the Synthesis of NO-Donating Derivatives of Stilbenes, Polyphenols and Flavonoids

It will be readily apparent to one skilled in the art that numerous methods exist for the synthesis of nitric oxide donating analogues or derivatives of stilbenes, such as resveratrol, polyphenols, or flavonoids, such as naringenin, or of other anti-oxidant, serum cholesterol decreasing or reverse cholesterol transport activating or HDL increasing compounds. Despite the existence of known methods, no such compounds have ever been described or synthesized before. Preferably, such compounds would be analogues or derivatives of stilbenes, such as resveratrol, of polyphenols, or of flavonoids, such as naringenin, or of other anti-oxidant, serum cholesterol decreasing or reverse cholesterol transport activating or HDL increasing compounds bound to nitric oxide donating moieties. Most preferably, such compounds would be analogues or derivatives of stilbenes, such as resveratrol, polyphenols, or flavonoids, such as naringenin, or of other anti-oxidant, serum cholesterol decreasing or reverse cholesterol transport activating or of HDL increasing compounds with one or more ONO.sub.2 groups, also referred to as nitric esters, organic nitrates, or nitrooxy groups, replacing hydroxyl groups of the parent compound.

An example of such a compound provided for by the present invention is resveratrol substituted with organic nitrate groups in place of the three hydroxyl groups present on naturally occurring resveratrol. This compound would be named 3,4′,5 trinitrooxy trans stilbene, or resveratrol tri nitrate, or using IUPAC nomenclature, 1,3-BIS-nitrooxy-5-[2-(4-nitrooxy-phenyl)-vinyl)-benzene. Another example of such a compound provided for by the present invention is naringenin substituted with organic nitrate groups in place of the three hydroxyl groups present on naturally occurring naringenin. This compound would be named naringenin trinitrate, or using IUPAC nomenclature, 5,7-bis-nitrooxy-2-(4-nitrooxy-phenyl)-chroman-4-one. While not being limited to those compounds explicitly described herein, many more examples are provided in the example section of the present invention.

The trans-resveratrol source material to be used in the reaction could be obtained commercially from Bio-Stat Limited (Stockport, U.K.) or Sigma Chemical Co. (St. Louis, Mo., USA), isolated from wine using the procedure of Goldberg et al. (1995) Am. J. Enol. Vitic. 46(2):159-165. Alternatively, trans-resveratrol may be synthesized according to the method of Toppo as taught in U.S. Pat. No. 6,048,903 or from appropriately substituted phenols by means of a Wittig reaction modified by Waterhouse from the method of Moreno-Manas and Pleixats.

The naringenin to be used as an ingredient for synthesis reactions is a naturally occurring compound readily available from numerous commercial sources, or alternatively, isolatable using well known methods requiring no undue experimentation from natural sources such as citrus juice.

Administration

For treatment of the conditions referred to above the compounds may be used per se, but more preferably are presented with an acceptable carrier or excipient in the form of a pharmaceutically acceptable formulation. These formulations include those suitable for oral, rectal, topical, buccal and parenteral (e.g. subcutaneous, intramuscular, intradermal, or intravenous) administration, although the most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of the compound as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association the active compound and the carrier or excipient (which may constitute one or more accessory ingredients). The carrier must be acceptable in the sense of being compatible with the other ingredients of the formulation and must not be deleterious to the recipient. The carrier may be a solid or a liquid, or both, and is preferably formulated with the compound as a unit-dose formulation, for example, a tablet, which may contain from 0.05% to 95% by weight of the active compound. Other pharmacologically active substances may also be present including other compounds. The formulations of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. In general, suitable formulations may be advantageously prepared by uniformly and intimately admixing the active compound with a liquid or finely divided solid carrier, or both, and then, if necessary, shaping the product. For example, a tablet may be prepared by compressing or molding a powder or granules of the compound, optionally with one or more assessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Formulations suitable for buccal (sub-lingual) administration include lozenges comprising a compound in a flavored base, usually sucrose and atacia or tragacanth, and pastilles comprising the compound in an inert base such as gelatin and glycerin or sucrose and acacia.

Formulations of the present invention suitable for parenteral administration comprise sterile aqueous preparations of the compounds, which are approximately isotonic with the blood of the intended recipient. These preparations are administered intravenously, although administration may also be effected by means of subcutaneous, intramuscular, or intradermal injection. Such preparations may conveniently be prepared by admixing the compound with water and rendering the resulting solution sterile and isotonic with the blood. Injectable compositions according to the invention will generally contain from 0.1 to 5% w/w of the active compound.

Formulations suitable for rectal administration are presented as unit-dose suppositories. These may be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers and excipients which may be used include Vaseline, lanoline, polyethylene glycols, alcohols, and combinations of two or more thereof. The active compound is generally present at a concentration of from 0.1 to 15% w/w of the composition, for example, from 0.5 to 2%.

The amount of active compound administered will, of course, be dependent on the subject being treated, the subject's weight, the manner of administration and the judgment of the prescribing physician. In the method of the invention a dosing schedule will generally involve the daily or semi-daily administration of the encapsulated compound at a perceived dosage of 1 ug to 1000 mg. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

### EXAMPLES

The following examples are set forth to assist in understanding the invention and should not be construed as specifically limiting the invention described and claimed herein. Such variations of the inventions which would be within the purview of those skilled in the art, including the substitution of equivalent compounds now known or later developed, including changes in formulation or minor changes in experimental design, are to be considered to fall within the scope of the invention incorporated herein.

For all the examples provided herein, unless otherwise noted the term “the compounds” or “the compound” will refer to any of the compounds provided for in the present invention.

### Example 1

### Preparation of 1,3-BIS-nitrooxy-5-[2-(4-nitrooxy-phenyl)-vinyl)-benzene.

To a solution of 1 mmol of 5-[(E)-2-(4-hydroxy-phenyl)-vinyl]-benzene-1,3-diol (synonym: resveratrol; 3,4′,5 trihydroxy trans stilbene) in 5 ml of dry THF at 25° C. is added 3 mmol of SOCl(NO.sub.3) or SO(NO.sub.3).sub.2. After 1 hr, Et.sub.2O (diethyl ether) is added and the solution is washed with water, dried and evaporated. The fully nitrated product (1,3-BIS-nitrooxy-5-[(E)-2-(4-nitrooxy-phenyl)-vinyl)-benzene) and the partially nitrated products (wherein any of the hydroxyl groups are independently replaced by ONO.sub.2 groups) are purified and isolated by chromatography on silica gel.

### Example 2

### Preparation of Piceatannol Tetranitrate

To a solution of 1 mmol of 1,2-benzenediol, 4-(2-(3,5-dihydroxyphenyl)ethenyl)-(E)-(synonym: piceatannol) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product (piceatannol tetranitrate) and the partially nitrated products (wherein any of the hydroxyl groups are independently replaced by ONO.sub.2 groups) are purified and isolated by chromatography on silica gel.

### Example 3

### Preparation of Butein Tetranitrate

To a solution of 1 mmol of 3,4,2′,4′-tetrahydroxychalcone (synonym: butein) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product butein tetranitrate and the partially nitrated products (wherein any of the hydroxyl groups are independently replaced by ONO.sub.2 groups) are purified and isolated by chromatography on silica gel.

### Example 4

### Preparation of Isoliquiritigenin Trinitrate

To a solution of 1 mmol of 4,2′,4′-trihydroxychalcone (synonym: isoliquiritigenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product isoliquiritienin trinitrate and the partially nitrated products (wherein any of the hydroxyl groups are independently replaced by ONO.sub.2 groups) are purified and isolated by chromatography on silica gel.

### Example 5

### Preparation of Fisetin Tetranitrate

To a solution of 1 mmol of 3,7,3′,4′-tetrahydroxyflavone (synonym: fisetin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product fisetin tetranitrate and the partially nitrated products (wherein any of the hydroxyl groups are independently replaced by ONO.sub.2 groups) are purified and isolated by chromatography on silica gel.

### Example 6

### Preparation of Quercetin Pentanitrate

To a solution of 1 mmol of 3,5,7,3′,4′-pentahydroxyflavone (synonym: quercetin) in 5 ml of dry THF at 25° C. is added 5 mmol of SOCl(NO.SUB.3) or SO(NO.SUB.3).sub.2. After 1 hr, Et.sub.2O (diethyl ether) is added and the solution is washed with water, dried and evaporated. The fully nitrated product quercetin pentanitrate and the partially nitrated products (wherein any of the hydroxyl groups are independently replaced by ONO.sub.2 groups) are purified and isolated by chromatography on silica gel.

### Example 7

### Preparation of N-(3,5-Bis-nitrooxy-phenyl)-N′-(4-nitrooxy-phenyl)-hydrazine

To a solution of 1 mmol of 5-[N′-(4-hydroxy-phenyl)-hydrazino]-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product N-(3,5-Bis-nitrooxy-phenyl)-N′-(4-nitrooxy-phenyl)-hydrazine and the partially nitrated products (wherein any of the hydroxyl groups are independently replaced by ONO.sub.2 groups) are purified and isolated by chromatography on silica gel.

### Example 8

### Preparation of 1,3-bis-nitrooxy-5-(4-nitrooxy-phenyldisulfanyl)-benzene

To a solution of 1 mmol of 5-(4-hydroxy-phenyldisulfanyl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-bis-nitrooxy-5-(4-nitrooxy-phenyldisulfanyl)-benzene and the partially nitrated products (wherein any of the hydroxyl groups are independently replaced by ONO.sub.2 groups) are purified and isolated by chromatography on silica gel.

### Example 9

### Preparation of 1,3-bis-nitrooxy-5-(4-nitrooxy-phenylperoxy)-benzene

To a solution of 1 mmol of 5-(4-hydroxy-phenylperoxy)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-bis-nitrooxy-5-(4-nitrooxy-phenylperoxy)-benzene and the partially nitrated products (wherein any of the hydroxyl groups are independently replaced by ONO.sub.2 groups) are purified and isolated by chromatography on silica gel.

### Example 10

### Preparation of 1,3-bis-nitrooxy-5-(4-nitrooxy-phenylsulfanylmethyl)-benzene

To a solution of 1 mmol of 5-(4-hydroxy-phenylsulfanylmethyl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-bis-nitrooxy-5-(4-nitrooxy-phenylsulfanylmethyl)-benzene and the partially nitrated products (wherein any of the hydroxyl groups are independently replaced by ONO.sub.2 groups) are purified and isolated by chromatography on silica gel.

### Example 11

### Preparation of N-(3,5-bis-nitrooxy-phenyl-O-(4-nitrooxy-phenyl)-hydroxylamine

To a solution of 1 mmol of 5-(4-hydroxy-phenoxyamino)-benzene-1,3-diol in 5 ml of dry TBF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product N-(3,5-bis-nitrooxy-phenyl-O-(4-nitrooxy-phenyl)-hydroxylamine and the partially nitrated products (wherein any of the hydroxyl groups are independently replaced by ONO.sub.2 groups) are purified and isolated by chromatography on silica gel.

### Example 12

### Preparation of benzyl-(4-nitrooxy-phenyl)-amine

To a solution of 1 mmol of 4-benzylamino-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product benzyl-(4-nitrooxy-phenyl)-amine is purified and isolated by chromatography on silica gel.

### Example 13

### Preparation of 2-(salicylideneamino)phenol dinitrate

To a solution of 1 mmol of 2-(salicylideneamino) phenol in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(salicylideneamino)phenol dinitrate and the partially nitrated products (wherein either of the hydroxyl groups are independently replaced by ONO.sub.2 groups) are purified and isolated by chromatography on silica gel.

### Example 14

### Preparation of (2,4-bis-nitrooxy-phenyl)-(2-nitrooxy-phenyl)-diazene

To a solution of 1 mmol of 4-(2-hydroxy-phenylazo)-benzene-1,3-diol (synonym: 1,3-benzenediol, 4-((2-hydroxyphenyl)azo)-) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2,4-bis-nitrooxy-phenyl)-(2-nitrooxy-phenyl)-diazene and the partially nitrated products (wherein any of the hydroxyl groups are independently replaced by ONO.sub.2 groups) are purified and isolated by chromatography on silica gel.

### Example 15

### Preparation of bis-(2,2′-nitrooxy-phenyl)-diazene

To a solution of 1 mmol of bis-(2,2′-hydroxy-phenyl)-diazene (synonym: 1-hydroxy-2-(2-hydroxyphenylazo)benzene) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product bis-(2,2′-nitrooxy-phenyl)-diazene and the partially nitrated products (wherein either of the hydroxyl groups are independently replaced by ONO.sub.2 groups) are purified and isolated by chromatography on silica gel.

### Example 16

### Preparation of N-(3-nitrooxy-phenyl)-benzenesulfonamide

To a solution of 1 mmol of N-(3-hydroxy-phenyl)-benzenesulfonamide (synonym: N-(3-hydroxyphenyl)benzene sulphonamide) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-(3-nitrooxy-phenyl)-benzenesulfonamide is purified and isolated by chromatography on silica gel.

### Example 17

### Preparation of N-(4-nitrooxy-phenyl)-benzenesulfonamide

To a solution of 1 mmol of N-(4-hydroxy-phenyl)-benzenesulfonamide (synonym: N-(4-hydroxyphenyl)benzene sulphonamide) in 5 ml of dry TMF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-(4-nitrooxy-phenyl)-benzenesulfonamide is purified and isolated by chromatography on silica gel.

### Example 18

### Preparation of 3,3′,4,5′-tetranitrooxybibenzyl

To a solution of 1 mmol of 3,3′,4,5′-tetrahydroxybibenzyl in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 3,3′,4,5′-tetranitrooxybibenzyl and the partially nitrated products (wherein any of the hydroxyl groups are independently replaced by ONO.sub.2 groups) are purified and isolated by chromatography on silica gel.

### Example 19

### Preparation of 1-benzyloxy-2-nitrooxy-benzene

To a solution of 1 mmol of 2-benzyloxy-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-benzyloxy-2-nitrooxy-benzene is purified and isolated by chromatography on silica gel.

### Example 20

### Preparation of benzoic acid 3-nitrooxy-phenyl ester

To a solution of 1 mmol of benzoic acid 3-hydroxy-phenyl ester (synonym: resorcinol monobenzoate) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product benzoic acid 3-nitrooxy-phenyl ester is purified and isolated by chromatography on silica gel.

### Example 21

### Preparation of 2-nitrooxy-benzoic acid phenyl ester

To a solution of 1 mmol of 2-hydroxy-benzoic acid phenyl ester (synonym: phenyl salicylate) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 2-nitrooxy-benzoic acid phenyl ester is purified and isolated by chromatography on silica gel.

### Example 22

### Preparation of 2-nitrooxy-N-(4-nitrooxy-phenyl)-benzamide

To a solution of 1 mmol of 2-hydroxy-N-(4-hydroxy-phenyl)-benzamide (synonym: Osalmid) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-nitrooxy-N-(4-nitrooxy-phenyl)-benzamide and the partially nitrated products (wherein either of the hydroxyl groups are independently replaced by ONO.sub.2 groups) are purified and isolated by chromatography on silica gel.

### Example 23

### Preparation of 2-nitrooxy-N-(3-nitrooxy-phenyl)-benzamide

To a solution of 1 mmol of 2-hydroxy-N-(3-hydroxy-phenyl)-benzamide in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-nitrooxy-N-(3-nitrooxy-phenyl)-benzamide and the partially nitrated products (wherein either of the hydroxyl groups are independently replaced by ONO.sub.2 groups) are purified and isolated by chromatography on silica gel.

### Example 24

### Preparation of 3,4,5-tris-nitrooxy-N-phenyl-benzamide

To a solution of 1 mmol of 3,4,5-trihydroxy-N-((Z)-1-methylene-but-2-enyl)-benzamide (synonym: gallanilide) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 3,4,5-tris-nitrooxy-N-phenyl-benzamide and the partially nitrated products (wherein any of the hydroxyl groups are independently replaced by ONO.sub.2 groups) are purified and isolated by chromatography on silica gel.

### Example 25

### Preparation of 1-(2,4-bis-nitrooxy-phenyl)-2-phenyl-ethanone

To a solution of 1 mmol of 1-(2,4-hydroxy-phenyl)-2-phenyl-ethanone (synonym: benzyl 2,4-dihydroxyphenyl ketone) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-(2,4-bis-nitrooxy-phenyl)-2-phenyl-ethanone and the partially nitrated products (wherein either of the hydroxyl groups are independently replaced by ONO.sub.2 groups) are purified and isolated by chromatography on silica gel.

### Example 26

### Preparation of 1,2-bis-nitrooxy-3-phenoxy-benzene

To a solution of 1 mmol of 3-phenoxy-benzene-1,2-diol in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,2-bis-nitrooxy-3-phenoxy-benzene and the partially nitrated products (wherein either of the hydroxyl groups are independently replaced by ONO.sub.2 groups) are purified and isolated by chromatography on silica gel.

### Example 27

### Preparation of 1,2-bis-nitrooxy-3-(2-nitrooxy-phenoxy)-benzene

To a solution of 1 mmol of 3-(2-hydroxy-phenoxy)-benzene-1,2-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,2-bis-nitrooxy-3-(2-nitrooxy-phenoxy)-benzene and the partially nitrated products (wherein any of the hydroxyl groups are independently replaced by ONO.sub.2 groups) are purified and isolated by chromatography on silica gel.

### Example 28

### Preparation of 1-nitrooxy-2-phenoxy-benzene

To a solution of 1 mmol of 2-phenoxy-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-2-phenoxy-benzene is purified and isolated by chromatography on silica gel.

### Example 29

### Preparation of 5,5 sulphinyl bis resorcinol tetranitrate

To a solution of 1 mmol of 5,5 sulphinyl bis resorcinol in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,5 sulphinyl bis resorcinol tetranitrate and the partially nitrated products (wherein any of the hydroxyl groups are independently replaced by ONO.sub.2 groups) are purified and isolated by chromatography on silica gel.

### Example 30

### Preparation of 1,3-benzenediol 4,4′-thiobis tetranitrate

To a solution of 1 mmol of 1,3-benzenediol 4,4′-thiobis in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1,3-benzenediol 4,4′-thiobis tetranitrate and the partially nitrated products (wherein any of the hydroxyl groups are independently replaced by ONO.sub.2 groups) are purified and isolated by chromatography on silica gel.

### Example 31

### Preparation of phenol 2,2′ thiobis dinitrate

To a solution of 1 mmol of phenol 2,2′ thiobis in 5 ml of dry THF at 25° C. is added 2 mmol of SOCl(NO.SUB.3) or SO(NO.SUB.3).sub.2. After I hr, Et.sub.2O (diethyl ether) is added and the solution is washed with water, dried and evaporated. The fully nitrated product phenol 2,2′ thiobis dinitrate and the partially nitrated products (wherein either of the hydroxyl groups are independently replaced by ONO.sub.2 groups) are purified and isolated by chromatography on silica gel.

### Example 32

### Preparation of 1-benzyl-2,4-bis-nitrooxy-benzene

To a solution of 1 mmol of 4-benzyl-benzene-1,3-diol (synonym: 1,3 benzenediol 3-phenyl methyl) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-benzyl-2,4-bis-nitrooxy-benzene and the partially nitrated products (wherein either of the hydroxyl groups are independently replaced by ONO.sub.2 groups) are purified and isolated by chromatography on silica gel.

### Example 33

### Preparation of 2-benzyl-1,4-bis-nitrooxy-benzene

To a solution of 1 mmol of 2-benzyl-benzene-1,4-diol (synonym: 1,4 benzenediol 4-phenyl methyl) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-benzyl-1,4-bis-nitrooxy-benzene and the partially nitrated products (wherein either of the hydroxyl groups are independently replaced by ONO.sub.2 groups) are purified and isolated by chromatography on silica gel.

### Example 34

### Preparation of (2,3,4-tris-nitrooxy-phenyl)-(3,4,5-tris-nitrooxy-phenyl)-methanone

To a solution of 1 mmol of (2,3,4-trihydrooxy-phenyl)-(3,4,5-trihydroxy-phenyl)-methanone (synonym: Exifone) in 5 ml of dry THF at 25° C. is added 6 mmol of SOCl(NO.SUB.3) or SO(NO.SUB.3).sub.2. After 1 hr, Et.sub.2O (diethyl ether) is added and the solution is washed with water, dried and evaporated. The fully nitrated product (2,3,4-tris-nitrooxy-phenyl)-(3,4,5-tris-nitrooxy-phenyl)-methanone and the partially nitrated products (wherein any of the hydroxyl groups are independently replaced by ONO.sub.2 groups) are purified and isolated by chromatography on silica gel.

### Example 35

### Preparation of (2-nitrooxy-phenyl)-phenyl-amine

To a solution of 1 mmol of 2-phenylamino-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product (2-nitrooxy-phenyl)-phenyl-amine is purified and isolated by chromatography on silica gel.

### Example 36

### Preparation of 2-(3,5-bis-nitrooxy-phenyl)-6-nitrooxy-4H-chromene

To a solution of 1 mmol of 5-(6-hydroxy-4H-chromen-2-yl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,5-bis-nitrooxy-phenyl)-6-nitrooxy-4H-chromene and the partially nitrated products (wherein any of the hydroxyl groups are independently replaced by ONO.sub.2 groups) are purified and isolated by chromatography on silica gel.

### Example 37

### Preparation of 2-(3,5-bis-nitrooxy-phenyl)-6-nitrooxy-1,4-dihydro-naphthalene

To a solution of 1 mmol of 5-(6-hydroxy-1,4-dihydro-naphthalen-2-yl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,5-bis-nitrooxy-phenyl)-6-nitrooxy-1,4-dihydro-naphthalene and the partially nitrated products (wherein any of the hydroxyl groups are independently replaced by ONO.sub.2 groups) are purified and isolated by chromatography on silica gel.

### Example 38

### Preparation of 2-(3,5-bis-nitrooxy-phenyl)-6-nitrooxy-1,2,3,4-tetrahydro-naphthalene

To a solution of 1 mmol of 5-(6-hydroxy-1,2,3,4-tetrahydro-naphthalen-2-yl)-benzene-1,3-diol in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,5-bis-nitrooxy-phenyl)-6-nitrooxy-1,2,3,4-tetrahydro-naphthalene and the partially nitrated products (wherein any of the hydroxyl groups are independently replaced by ONO.sub.2 groups) are purified and isolated by chromatography on silica gel.

### Example 39

### Preparation of 5,7-bis-nitrooxy-2-(4-nitrooxy-phenyl)-chroman-4-one

To a solution of 1 mmol of 5,7-dihydroxy-2-(4-hydroxy-phenyl)-chroman-4-one (Synonym: naringenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-2-(4-nitrooxy-phenyl)-chroman-4-one and the partially nitrated products (wherein any of the hydroxyl groups are independently replaced by ONO.sub.2 groups) are purified and isolated by chromatography on silica gel.

### Example 40

### Preparation of 5,7-bis-nitrooxy-2-(4-nitrooxy-phenyl)-chromen-4-one

To a solution of 1 mmol of 5,7-dihydroxy-2-(4-hydroxy-phenyl)-chromen-4-one (Synonym: apigenin) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-2-(4-nitrooxy-phenyl)-chromen-4-one and the partially nitrated products (wherein any of the hydroxyl groups are independently replaced by ONO.sub.2 groups) are purified and isolated by chromatography on silica gel.

### Example 41

### Preparation of 5,7-bis-nitrooxy-3-(4-nitrooxy-phenyl)-chromen-4-one

To a solution of 1 mmol of 5,7-dihydroxy-3-(4-hydroxy-phenyl)-chromen-4-one (Synonym: genistein) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,7-bis-nitrooxy-3-(4-nitrooxy-phenyl)-chromen-4-one and the partially nitrated products (wherein any of the hydroxyl groups are independently replaced by ONO.sub.2 groups) are purified and isolated by chromatography on silica gel.

### Example 42

### Preparation of 2-(3,4-bis-nitrooxy-phenyl)-3,4,5,7-tetrakis-nitrooxy-chroman

To a solution of 1 mmol of 2-(3,4-dihydroxy-phenyl)-chroman-3,4,5,7-tetraol (synonym: leucocianidol) in 5 ml of dry THF at 25° C. is added 6 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,4-bis-nitrooxy-phenyl)-3,4,5,7-tetrakis-nitrooxy-chroman and the partially nitrated products (wherein any of the hydroxyl groups are independently replaced by ONO.sub.2 groups) are purified and isolated by chromatography on silica gel.

### Example 43

### Preparation of 6-hydroxy-7-nitrooxy-3-(4-nitrooxy-phenyl)-chroman-4-one

To a solution of 1 mmol of 6,7-dihydroxy-3-(4-hydroxy-phenyl)-chroman-4-one (Synonym: 6,7,4′-trihydroxyisoflavanone) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 6-hydroxy-7-nitrooxy-3-(4-nitrooxy-phenyl)-chroman-4-one and the partially nitrated products (wherein any of the hydroxyl groups are independently replaced by ONO.sub.2 groups) are purified and isolated by chromatography on silica gel.

### Example 44

### Preparation of Quracol B Tetranitrate

To a solution of 1 mmol of Quracol B in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product Quracol B tetranitrate and the partially nitrated products (wherein any of the hydroxyl groups are independently replaced by ONO.sub.2 groups) are purified and isolated by chromatography on silica gel.

### Example 45

### Preparation of 1-(4-hydroxy-2,6-bis-nitrooxy-phenyl)-3-(4-nitrooxy-phenyl)-propan-1-one

To a solution of 1 mmol of 3-(4-hydroxy-phenyl)-1-(2,4,6-trihydroxy-phenyl)-propan-1-one (Synonym: phloretin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 1-(4-hydroxy-2,6-bis-nitrooxy-phenyl)-3-(4-nitrooxy-phenyl)-propan-1-one and the partially nitrated products (wherein any of the hydroxyl groups are independently replaced by ONO.sub.2 groups) are purified and isolated by chromatography on silica gel.

### Example 46

### Preparation of 1-nitrooxy-4-((Z)-3-phenyl-allyl)-benzene

To a solution of 1 mmol of 4-((Z)-3-phenyl-allyl)-phenol (synonym: 4(-3-phenyl-2-propenyl)-,(E)-phenol) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-4-((Z)-3-phenyl-allyl)-benzene is purified and isolated by chromatography on silica gel.

### Example 47

### Preparation of 1-nitrooxy-4-((E)-3-phenyl-propenyl)-benzene

To a solution of 1 mmol of 4-((E)-3-phenyl-propenyl)-phenol in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 1-nitrooxy-4-((E)-3-phenyl-propenyl)-benzene is purified and isolated by chromatography on silica gel.

### Example 48

### Preparation of 5,6,7-tris-nitrooxy-2-phenyl-chromen-4-one

To a solution of 1 mmol of 5,6,7-trihydroxy-2-phenyl-chromen-4-one (synonym: baicalein) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5,6,7-tris-nitrooxy-2-phenyl-chromen-4-one and the partially nitrated products (wherein any of the hydroxyl groups are independently replaced by ONO.sub.2 groups) are purified and isolated by chromatography on silica gel.

### Example 49

### Preparation of Rutin Tetranitrate

To a solution of 1 mmol of 2-(3,4-dihydroxy-phenyl)-5,7-dihydroxy-3-[(2S,3R,5S,6R)-3,4,5-trihydroxy-6-((2R,3R,4R,5R,6S)-3,4,5-trihydroxy-6-methyl-tetrahydro-pyran-2-yloxymethyl)-tetrahydro-pyran-2-yloxy]-chromen-4-one (Synonym: rutin) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product 2-(3,4-bis-nitrooxy-phenyl)-5,7-bis-nitrooxy-3-[(2S,3R,5S ,6R)-3,4,5-trihydroxy-6 ((2R,3R,4R,5R,6S)-3,4,5-trihydroxy-6-methyl-tetrahydro-pyran-2-yloxymethyl)-tetrahydro-pyran-2-yloxy]-chromen-4-one (rutin tetranitrate) and the partially nitrated products (wherein any of the hydroxyl groups are independently replaced by ONO.sub.2 groups) are purified and isolated by chromatography on silica gel.

### Example 50

### Preparation of 5-hydroxy-2-(4-hydroxyphenyl)-7-(2-O-alpha-L-rhamnopyranosyl-beta-D-glucopyranosyloxy)-4-chromanon dinitrate

To a solution of 1 mmol of 5-hydroxy-2-(4-hydroxyphenyl)-7-(2-O-alpha-L-rhamnopyranosyl-beta-D-glucopyranosyloxy)-4-chromanon (synonym: naringin) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5-hydroxy-2-(4-hydroxyphenyl)-7-(2-O-alpha-L-rhamnopyranosyl-beta-D-glucopyranosyloxy)-4-chromanon dinitrate and the partially nitrated products (wherein either of the hydroxyl groups are independently replaced by ONO.sub.2 groups) are purified and isolated by chromatography on silica gel.

### Example 51

### Preparation of (E)-(3S,5R)-7-[3-(4-fluoro-phenyl)-1-isopropyl-1H-indol-2-yl]-1,3,5-tris-nitrooxy-hept-6-en-1-one

To a solution of 1 mmol of (E)-(3S,5R)-7-[3-(4-fluoro-phenyl)-1-isopropyl-1H-indol-2-yl]-3,5-dihydroxy-hept-6-enoic acid (Synonym: fluvastatin; Novartis) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product (E)-(3S,5R)-7-[3-(4-fluoro-phenyl)-1-isopropyl-1H-indol-2-yl]-1,3,5-tris-nitroxy-hept-6-en-1-one and the partially nitrated products (wherein any of the hydroxyl groups are independently replaced by ONO.sub.2 groups) are purified and isolated by chromatography on silica gel.

### Example 52

### Preparation of 5-(4-fluoro-phenyl)-2-isopropyl-4-phenyl-1-((3R,5R)-3,5,7-tris-nitrooxy-7-oxo-heptyl)-1H-pyrrol-1-yl]-3-carboxylic acid phenylamide

To a solution of 1 mmol of (3R,5R)-7-[2-(4-fluoro-phenyl)-5-isopropyl-3-phenyl4-phenylcarbamoyl-pyrrol-1-yl]-3,5-dihydroxy-heptanoic acid (Synonym: atorvastatin; Parke-Davis) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product 5-(4-fluoro-phenyl)-2-isopropyl-4-phenyl-1-((3R,5R)-3,5,7-tris-nitrooxy-7-oxo-heptyl)-1H-pyrrol-1-yl]-3-carboxylic acid phenylamide and the partially nitrated products (wherein any of the hydroxyl groups are independently replaced by ONO.sub.2 groups) are purified and isolated by chromatography on silica gel.

### Example 53

### Preparation of (E)-(3R,5S)-7-[4-(4-fluoro-phenyl)-2,6-diisopropyl-5-methoxymethyl-pyridin-3-yl]1,3,5-tris-nitrooxy-hept-6-en-1-one

To a solution of 1 mmol of (E)-(3R,5S)-7-[4-(4-fluoro-phenyl)-2,6-diisopropyl-5-methoxymethyl-pyridin-3-yl]-3,5-dihydroxy-hept-6-enoic acid (Synonym: cerivastatin; Bayer) in 5 ml of dry THF at 25° C. is added 3 mmol of SOCl(NO.SUB.3) or SO(NO.SUB.3).sub.2. After 1 hr, Et.sub.2O (diethyl ether) is added and the solution is washed with water, dried and evaporated. The fully nitrated product (E)-(3R,5S)-7-[4-(4-fluoro-phenyl)-2,6-diisopropyl-5-methoxymethyl-pyridin-3-yl]-1,3,5-tris-nitrooxy-hept-6-en-1-one and the partially nitrated products (wherein any of the hydroxyl groups are independently replaced by ONO.sub.2 groups) are purified and isolated by chromatography on silica gel.

### Example 54

### Preparation of (S)-2-methyl-butyric acid (1S,3S,7S,8S,8aR)-7-methyl-3-nitrooxy-8-((4R,6R)-3,5,7-tris-nitrooxy-7-oxo-heptyl)-1,2,3,7,8,8a-hexahydro-napthalen-1-yl ester

To a solution of 1 mmol of (2R,4R)-3,5-dihydroxy-7-[(1S,2S,6S,8S,8aR)-6-hydroxy-2-methyl-8-((S)-2-methyl-butyryloxy)-1,2,6,7,8,8a-hexahydro-napthalen-1-yl]-heptanoic acid (Synonym: pravastatin; Bristol-Myers Squibb) in 5 ml of dry THF at 25° C. is added 4 mmol of SOCl(NO.SUB.3) or SO(NO.SUB.3).sub.2. After 1 hr, Et.sub.2O (diethyl ether) is added and the solution is washed with water, dried and evaporated. The fully nitrated product (S)-2-methyl-butyric acid (1S,3S,7S,8S,8aR)-7-methyl-3-nitrooxy-8-((4R,6R)-3,5,7-tris-nitrooxy-7-oxo-heptyl)-1,2,3,7,8,8a-hexahydro-napthalen-1-yl ester and the partially nitrated products (wherein any of the hydroxyl groups are independently replaced by ONO.sub.2 groups) are purified and isolated by chromatography on silica gel.

### Example 55

### Preparation of 2,2-dimethyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester

To a solution of 1 mmol of 2,2-dimethyl-butyric acid (1S,3R,7S,8S,8aR)-8-[2-((2R,4R)-4-hydroxy-6-oxo-tetrahydro-pyran-2-yl)-ethyl]-3,7-dimethyl-1,2,3,7,8,8a-hexahydro-napthalen-1-yl ester (synonym: simvastatin; Merck) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product 2,2-dimethyl-butyric acid (1S ,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester is purified and isolated by chromatography on silica gel.

### Example 56

### Preparation of (S)-2-methyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester

To a solution of 1 mmol of (S)-2-methyl-butyric acid (1S,3R,7S,8S,8aR)-8-[2-((2R,4)-4-hydroxy-6-oxo-tetrahydro-pyran-2-yl)-ethyl]-3,7-dimethyl-1,2,3,7,8,8a-hexahydro-napthalen-1-yl ester (synonym: lovastatin; Merck) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product (S)-2-methyl-butyric acid (1S,3R,7S,8S,8aR)-3,7-dimethyl-8-[2-((2R,4R)-4-nitrooxy-6-oxo-tetrahydro-pyran-2-yl)-ethyl]-1,2,3,7,8,8a-hexahydro-napthalen-1-yl ester is purified and isolated by chromatography on silica gel.

### Example 57

### Preparation of N-[4-(4-fluoro-phenyl)-6-isopropyl-5-((E)-(3R,5R)-3,5,7-tris-nitrooxy-7-oxo-hept-1-enyl)-pyrimidin-2-yl]-N-methyl-methanesulfonamide

To a solution of 1 mmol of (E)-(3R,5R)-7-[4-(4-fluoro-phenyl)-6-isopropyl-2-(methanesulfonyl-methyl-amino)-pyrimidin-5-yl]-3,5-dihydroxy-hept-6-enoic acid (synonym: rosuvastatin; Astra-Zeneca) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product N-[4-(4-fluoro-phenyl)-6-isopropyl-5-((E)-(3R,5R)-3,5,7-tris-nitrooxy-7-oxo-hept-1-enyl)-pyrimidin-2-yl]-N-methyl-methanesulfonamide is purified and isolated by chromatography on silica gel.

### Example 58

### Preparation of Nitrooxy-pyridin-3-yl-methanone

To a solution of 1 mmol of nicotinic acid (synonym: niacin) in 5 ml of dry THF at 25° C. is added 1 mmol of SOCl(NO.SUB.3) or SO(NO.SUB.3).sub.2. After 1 hr, Et.sub.2O (diethyl ether) is added and the solution is washed with water, dried and evaporated. The nitrated product nitrooxy-pyridin-3-yl-methanone is purified and isolated by chromatography on silica gel.

### Example 59

### Preparation of (S)-1-(4-fluoro-phenyl)-3-[(S)-3-(4-fluoro-phenyl)-3-nitrooxy-propyl]-4-(4-nitrooxy-phenyl)-azetidin-2-one

To a solution of 1 mmol of (S)-1-(4-fluoro-phenyl)-3-[(S)-3-(4-fluoro-phenyl)-3-hydroxy-propyl]-4-(4-hydroxy-phenyl)-azetidin-2-one (synonym: ezetimibe; Merck) in 5 ml of dry THF at 25° C. is added 2 mmol of SOCl(NO.SUB.3) or SO(NO.SUB.3).sub.2. After 1 hr, Et.sub.2O (diethyl ether) is added and the solution is washed with water, dried and evaporated. The fully nitrated product (S)-1-(4-fluoro-phenyl)-3-[(S)-3-(4-fluoro-phenyl)-3-nitrooxy-propyl]-4-(4-nitrooxy-phenyl)-azetidin-2-one and the partially nitrated products (wherein either of the hydroxyl groups are independently replaced by ONO.sub.2 groups) are purified and isolated by chromatography on silica gel.

### Example 60

### Method for Glucoronidating Compounds of the Invention

This example describes the method of preparing glucoronidated compounds of the invention. In this specific example, a dinitrated version of resveratrol, 3,4′-nitrooxy-5-hydroxy resveratrol (50-1000 μM) prepared as in Example 1 and 10 μl of human intestinal, 25 μl of colon or 10 μl of liver microsomes (200, 400, 200 μg of protein, respectively), 20 of μl recombinant UDP-glucuronosyltransferase (400 μg of protein) in a final volume of 500 μl of 50 mM Tris HCl buffer (pH 7.8) with 10 mM MgCl₂ are preincubated for 5 min at 37° C. The reactions are initiated by the addition of 1 mM 5′-diphosphoglucuronic acid. The reaction mixtures are incubated at 37° C. for 60 min. The samples are cooled on ice and subjected to solid-phase extraction using oasis Hydrophilic-Lipophilic Balance 1 cc C₁₈ extraction cartridges (Waters Corp, Milford, Mass.). The cartridges are washed with 1-ml methanol and equilibrated with 1-ml water. After loading 0.5 ml of the sample, the cartridges are washed with 5% methanol and eluted with 2 ml of 100% methanol. The methanol eluate is dried under N₂ gas at 40° C., and the sample is redissolved in 250 μl of mobile phase for HPLC analysis.

### Example 61

### Method for Sulfating Compounds of the Invention

This example describes the method of preparing sulfated compounds of the invention. In this specific example, a dinitrated version of resveratrol, 3,4′-nitrooxy-5-hydroxy resveratrol prepared as in Example 1 is sulfated by a sulfotransferase enzyme using a previously described ion-pair extraction method (Varin et al. 1987. Anal. Biochem. 161:176-180). The typical reaction mixture contains 0.1 to 200 μM of 3,4′-nitrooxy-5-hydroxy resveratrol, 1 μM [³⁵S]PAPS and 2.5 μl of pooled human liver cytosol (50 μg of protein), 2.5 μl of human jejunal cytosol (30 μg), Caco-2 cytosol (225 μg) or 0.25 μl recombinant sulfotransferase in 33 mM Tris-HCl buffer, pH 7.4, with 8 mM dithiothreitol and 0.0625% bovine serum albumin in a total volume of 100 μl. The samples are incubated for 30 min at 37° C., and the reactions terminated by the addition of 10 μl 2.5% acetic acid, 20 μl of 0.1 μM tetrabutylammonium hydrogen sulfate and 500 μl of ethyl acetate. After through mixing and centrifugation, 400 μl of the ethyl acetate extract is subjected to liquid scintillation counting after the addition of biodegradable counting scintillant (Amersham Biosciences, Piscataway, N.J.).

### Example 62

### Determination of ACAT Inhibition

The activity of compounds of the invention as inhibitors of ACAT may be determined by known methods, for example those taught in U.S. Pat. No. 6,165,984 and summarized below.

First, rats are sacrificed by decapitation and the livers excised. 1 g each of the livers is homogenized in 5 ml of homogenization medium (0.1 M KH.sub.2 PO.sub.4, pH 7.4, 0.1 mM EDTA and 10 mM .beta.-mercaptoethanol). The homogenate is centrifuged at 3,000.times.g for 10 min. at 4.degree. C. and the supernatant thus obtained is centrifuged at 15,000.times.g for 15 min. at 4.degree. C. to obtain a supernatant. The supernatant is put into an ultracentrifuge tube (Beckman) and centrifuged at 100,000.times.g for 1 hour at 4.degree. C. to obtain microsomal pellets, which are then suspended in 3 ml of the homogenization medium and centrifuged at 100,000.times.g for 1 hour at 4.degree. C. The pellets thus obtained are suspended in 1 ml of the homogenization medium. The concentration of proteins in the resulting suspension is determined by Lowry's method and then adjusted to 4 to 8 mg/ml. The resulting suspension is stored in a deep freezer (Biofreezer, Forma Scientific Inc.).

6.67 .mu.1 of 1 mg/ml cholesterol solution in acetone is mixed with 6 .mu.l of 10% Triton WR-1339(Sigma Co.) in acetone and, then, acetone is removed from the mixture by evaporation using nitrogen gas. Distilled water is added to the resulting mixture in an amount to adjust the concentration of cholesterol to 30 mg/ml.

To 10 .mu.l of the resulting aqueous cholesterol solution is added 10 .mu.l of 1 M KH.sub.2 PO.sub.4 (pH 7.4), 5 .mu.l of 0.6 mM bovine serum albumin (BSA), 10 .mu.l of microsome solution obtained in (Step 1) and 55 .mu.l of distilled water (total 90 .mu.l). The mixture is pre-incubated in a waterbath at 37.degree. C. for 30 min.

10 .mu.l of (1-.sup.14 C) oleoyl-CoA solution (0.05 .mu.Ci, final concentration: 10 .mu.M) is added to the pre-incubated mixture and the resulting mixture is incubated in a waterbath at 37.degree. C. for 30 min. To the mixture is added 500 .mu.l of isopropanol:heptane mixture (4:1(v/v)) 300 .mu.l of heptane and 200 .mu.l of 0.1 M KH.sub.2 PO.sub.4 (pH 7.4), and the mixture is mixed violently by using a vortex and then allowed to stand at a room temperature for 2 min.

200 .mu.l of the resulting supernatant is put in a scintillation bottle and 4 ml of scintillation fluid (Lumac) is added thereto. The mixture is assayed for radioactivity with liquid scintillation counter. ACAT activity is calculated as picomoles of cholesteryl oleate synthesized per min. per mg protein (pmoles/min/mg protein). ACAT activities observed rat groups that have received compounds of the invention are lower than those of the control group.

### Example 63

### Determination of Inhibition of HMG-CoA Reductase

The potency of inhibition of HMG-CoA reductase by compounds of the invention may be determined using known methods, such as that taught in U.S. Pat. No. 5,877,208. That method is summarized below.

Rats are sacrificed by decapitation and the livers are excised and immediately placed in an ice-cold homogenization medium (50 mM KH.sub.2 PO.sub.4 (pH 7.0), 0.2M sucrose, 2 mM dithiothreitol (DTT)). The livers are homogenized in the homogenization medium (2 ml medium/g of the liver) with a Waring blendor for 15 sec. (three strokes with a motor-driven Teflon pestle in a Potter-Elvehjem type glass homogenizer). The homogenate is centrifuged at 15,000.times.g for 10 min. and the supernatant thus obtained is centrifuged at 100,000.times.g for 75 min. to obtain microsomal pellets, which are then resuspended in the homogenization medium containing 50 mM EDTA and centrifuged at 100,000.times.g for 60 min. The supernatant containing the microsome is used as an enzyme source.

The activity of HMG-CoA reductase is determined by employing radiolabeled 14C HMG-CoA, in accordance with the method of Shapiro et al. (Biochemica et Biophysica Acta, 370, 369-377(1974)) as follows.

The enzyme in the supernatant containing the microsome obtained in (Step 1) is activated at 37.degree. C. for 30 min. Added to a reaction tube is 20 .mu.l of HMG-CoA reductase assay buffer (0.25M KH.sub.2 PO.sub.4 (pH 7.0), 8.75 mM EDTA, 25 mM DTT, 0.45M KCl and 0.25 mg/ml BSA), 5 .mu.l of 50 mM NADPH, 5 .mu.l of radiolabeled 14C HMG-CoA (0.05 .mu.Ci/tube, final conc. 120 .mu.M), and 10 .mu.l of activated microsomal enzyme (0.03-0.04 mg), and the mixture is incubated at 37.degree. C. for 30 min. The reaction is terminated by adding 10 .mu.l of 6M HCl to the mixture, and the mixture is incubated at 37.degree. C. for 15 min. to allow complete lactonization of the product. The precipitate is removed by centrifugation at 10,000.times.g for 1 min. and the supernatant is applied to a Silica gel 60G TLC plate (Altech, Inc., Newark, U.S.A.) and then developed with benzene:acetone (1:1, v/v). The appropriate region is removed by scraping with a disposable cover slips and assayed for radioactivity with 1450 Microbeta liquid scintillation counter (Wallacoy, Finland). Enzyme activities are calculated as picomoles mevalonic acid synthesized per min. per mg protein (pmoles/min/mg protein). Control rats show a relatively high HMG-CoA reductase activity, while the HMG-CoA activities observed with rats fed compounds of the invention are lower than that of the control group.

### Example 64

### Determination of Activation of PPAR by Compounds of the Invention

The ability of compounds of the invention to modify the activity of PPARgamma and PPARalpha are determined by several known methods, such as those described below, which were previously taught in U.S. Pat. No. 6,369,098.

Method for Screening for Compounds that Modify the Activity of PPARgamma and PPARalpha Based on Inhibition of NF-kappaB Activation

Compounds of the invention are tested for the ability to inhibit activity of NF-kappaB. Human endothelial cells and vascular smooth muscle cells (VSMC) are known to express both PPARgamma and PPARalpha (Neve BP et al. Biochem Pharmacol., 60:1245-1250 (2000)). Alternatively, isolated human peripheral T lymphocytes from normal healthy donors or a mammalian cell line such as a Jurkat T cell line transfected with the PPARalpha and/or the PPARgamma expression vector may be used in these experiments. One of these selected cell types is stimulated with one or more of: phytohemagglutinin/phorbol-12-myristate-13-acetate (PHA/PMA), TNF-alpha, interferon-gamma or other factor that activates NF-kappaB. Activation of NF-kappaB is determined by electrophoretic mobility shift assay similar to that previously described (Rossi A et al. Proc Natl Acad Sci USA, 94:746-50 (1997)). Preincubation of the same cells with 5 micromolar of a compound of the invention 2 hours prior to addition of an activator of NF-kappaB inhibits the activation of NF-kappaB that is otherwise observed in the absence of the compound.

Method for Screening for Compounds that Modify the Activity of PPARgamma and PPARalpha Based on Inhibition of NFAT Activation

Isolated human peripheral T lymphocytes from normal healthy donors or a mammalian cell line such as a Jurkat T cell line transfected with the PPARalpha and/or the PPARgamma expression vector, are stimulated with one or more of PHA/PMA, TNF-alpha, interferon-gamma or other factor that activates NFAT. Transcriptional activation of NFAT is determined by electrophoretic mobility shift assay similar to that described by Yang et al. J Biol Chem.; 275:4541-4 (2000). Preincubation of the same cells with 5 micromolar of a compound of the invention for 2 hours prior to addition of an activator of NFAT inhibits the activation of NFAT otherwise observed in the absence of said compound.

Method for Screening for Compounds that Modify the Activity of PPARgamma and PPARapha Based on Inhibition of IL-2 Production

Isolated human T lymphocytes or a mammalian cell line such as a Jurkat T cell line transfected with a PPARalpha and/or a PPARgamma expression vector is stimulated with one or more of PHA/PMA, TNF-alpha, interferon-gamma or some other factor that activates induction of IL-2 gene expression. Production of IL-2 is determined measuring the concentration of IL-2 in the supernatant from cells using Endogen kits (Wolbum), as described by Yang et al. J Biol Chem., 275:4541-4 (2000). Preincubation of the same cells with 5 micromolar of a compound of the invention for 12 hours prior to addition of an activator of IL-2 production inhibits the activation of IL-2 production otherwise observed in the absence of said compound.

### Example 65

### Method of Determining the ABCA-1 Activating Ability of Compounds of the Invention

This test demonstrates the effectiveness of compounds of the invention on ABCA-1 gene expression, using a known method, as taught in U.S. Pat. No. 6,548,548. Briefly, the pGL3 luciferase reporter vector system (Promega, Madison, Wis.) is used to create a recombinant plasmid to measure reporter gene expression under control of the ABCA-1 promoter.

Plasmid pGL3-Basic (Promega, Madison, Wis.; Cat. #E1751) is used as a control plasmid containing the promoterless luciferase gene. The reporter construct containing the ABCA-1 promoter and luciferase gene is made by cloning a genomic fragment from the 5′ flanking region of the ABCA-1 gene (hAPR1 5′ promoter, corresponding to nucleotides 1080-1643 of SEQ ID NO: 3) into the SacI site of the GL3-Basic plasmid to generate plasmid GL-6a. Next, plasmid GL-6a is digested with SpeI and Acc65I. A BsiWI-SpeI fragment excised from a lambda subclone, representing the ABCA-1 genomic sequence corresponding to nucleotides 1-1534 of SEQ ID NO: 3 is ligated into the remaining vector/ABCA-I promoter fragment produced by this digestion. The resultant plasmid, pAPR1, encodes the luciferase reporter gene under transcriptional control of 1.75 kb of the human ABCA-1 promoter sequence.

The control or pAPR1 plasmid wisas transfected into confluent cultures of RAW 264.7 cells maintained in DMEM containing 10% fetal bovine serum. Each well of a 12 well dish is transfected for 5 hours with either pGL3-Basic, pGL3-Promoter or pAPR1 DNA (1 .mu.g), luciferase plasmid DNA (1 .mu.g), and 12 .mu.l of Geneporter reagent (Gene Therapy Systems, San Diego, Calif.; Cat. #T201007). In addition, 0.1 .mu.g of pCMV.beta. plasmid DNA (Clontech, Palo Alto, Calif., Cat. #6177-1) is added as a control for transfection efficiency. After 5 hours, the culture medium is replaced with serum-free DMEM/BSA in the presence or absence of acetylated LDL (100 .mu.g/ml) and incubated for 24 hours.

Following transfection, the cells in each well are lysed in 70 .mu.l of 1.times.cell lysis reagent (Promega, Madison, Wis., Cat. #E3971), subjected to one freeze-thaw cycle, and the lysate cleared by centrifugation for 5 minutes at 12,000 g. After centrifugation, 100 .mu.l of luciferase assay reagent (Promega, Madison, Wis.; Cat. #E1501) is added to 10 .mu.l of lysate. The luciferase activity of each lysate is measured as light units using a luminometer. Additionally, the .beta.-galactosidase activity of each lysate is measured using the chemiluminescent assay reagents supplied in the Galacto-light kit according to the manufacturer's instructions (Tropix Inc., Bedford, Mass.: Cat. #BL100G). The normalized luciferase activity for each lysate is determined by dividing the luciferase activity value by the determined .beta.-galactosidase value and reported as relative light units.

Compounds of the invention demonstrate increased ABCA-1 gene expression in this assay.

### Example 66

### Measurement of Human Apolipoprotein A1 Protein Expression

This study measures the effect of compounds of the invention on the level of apolipoprotein A1 protein expressed via the endogenous APO AI gene in CaCO2 cells, a human intestinal cell line, or in Hep G2 cells, a human hepatic cell line. Compounds of the invention are dissolved in appropriate solvent and then provided to CaCO2 or Hep G2 cells in cell culture media with serum and returned to a tissue culture incubator at 37° C. for 12, 24, 36 or 48 hours. Following rinsing of the cells with serum free media, the cells are fixed, lysed and the presence of apolipoprotein A1 detected with a commercially available human apolipoprotein A1 antibody (for example mouse anti-human apolipoprotein A1 antibody, Intracel Resources LLC, Frederick, Md., USA). The difference in the abundance of apolipoprotein A1 protein expression for cells treated with compounds of the invention relative to the abundance of expression in cells treated with solvent only is observed. The optimal concentration of each compound for the detection of its apolipoprotein expression modulating activity is determined by repeating the experiment with different concentrations of each compound ranging from about 0.1 picomolar up to about 100 millimolar in 2-fold concentration steps. Increased detection of antibody binding to cells reveals compounds that induce an increase in apolipoprotein A1 expression.

### Example 67

### Measurement of ApoA-1 Promoter Induction

CaCO2 or Hep G2 cells are exposed to effective concentrations of compounds of the invention. The cells are transfected, using a standard technique, with a reporter construct, pAI.474-Luc along with pRSV-Bgalactosidase, which monitors transfection efficiency. The pAI.474-Luc is a construct that was created using conventional molecular biology techniques and contains rat APO AI promoter nucleotides from −474 to −7 fused to the reporter gene, which is firefly luciferase (Luc) (U.S. patent application Ser. No. 10/222,013). Compounds of the invention are dissolved in appropriate solvent (for example, DMSO) and then added to the culture media for 16 hours. At the end of the treatment, the cells are harvested and the Luciferase activity is measured with a standard protocol employing a commercially available luciferase assay. Spent media exposed to the cells for 36 hours is also assayed for its content of APO AI protein using western blot analysis. Increased luciferase activity in the cell lysate or spent media indicates compounds of the invention with apolipoprotein Al expression inducing activity.

### Example 68

### Measurement of AGCCCCCGC Sequence Element Induction

CaCO2 or Hep G2 cells are exposed to effective concentrations of compounds of the invention. The cells are first transfected using standard techniques with a reporter construct comprising one or more copies of the nine nucleotides, 5′-AGCCCCCGC-3′ acting as an enhancer element (Kilbourne et al, JBC, 270(12):7004-7010, 1995), operably linked to a promoter (for example the thymidine kinase (TK) promoter), operably linked to a reporter gene (for example luciferase, CAT, or the apolipoprotein A1 gene) along with pRSV-Bgalactosidase, which monitors transfection efficiency (as taught in U.S. patent application Ser. No. 10/222,013). Compounds of the invention are then dissolved in appropriate solvent (for example, DMSO) and then added to the culture media for 16 hours. At the end of the treatment, the cells are harvested and the reporter gene activity is measured using standard assays that are commercially available. Increased or decreased reporter gene activity indicates that compounds of the invention have the ability to modulate transcription from promoters that contain the nine nucleotide sequence 5′-AGCCCCCGC-3′, which is believed to comprise an egr-1 response element. Compounds of the invention are therefore useful in the treatment of conditions, diseases or disorders associated with the activity of egr-1.

### Example 69

### Measurement of Vasodilation Activity of the Compounds Using a Ring Test

A standard isolated vascular ring preparation is used to establish potencies of the compounds provided for in the invention. Thoracic aortic rings from New Zealand White rabbits are suspended in pH 7.4 buffer at 37° C. and a 10 gram preload is applied to each. After a 2 hour equilibration, the rings are preconstricted with norepinephrine. Measuring the grams of relaxation induced by adding compounds of the invention to the organ baths at successively increasing concentrations, a dose-response curve is constructed for each compound. Sodium nitroprusside and glyceryl trinitrate are employed as positive controls.

Vasodilative activity is also determined in isolated rat aorta measuring the inhibition of the contraction induced by epinephrine in the tissue prepared in accordance with the method described by Reynolds et al. (J. Pharmacol. Exp. Therap. 252, 915, 1990).

Increased relaxation induced by the addition of compounds of the invention demonstrates the vasodilation activity and usefulness of the compounds for the treatment or prevention of numerous disorders associated with hypertension, for example cardiovascular disorders.

### Example 70

### Measurement of NO Donation

To demonstrate the utility of compounds of the invention as nitric oxide releasing agents, compounds of the invention are dissolved in an appropriate solvent and phosphate buffer at pH 7.4 and incubated in a 37° C. water bath. The NO release rate is measured periodically by flushing the solution with inert nitrogen gas and then sweeping newly generated NO into a chemiluminescence detector and integrating the signal produced over the next 4-7 minutes. Increased NO release relative to negative controls, potentially appropriate negative controls being for example hydroxylated rather than nitrated versions of the same compounds, demonstrates the NO releasing activity and usefulness as a treatment or prevention for disorders, disease or conditions associated with hypertension, for example cardiovascular disorders.

### Example 71

### Measurement of Antioxidant Effectiveness

The antioxidant performance of compounds of the invention is demonstrated by measuring the extent of low density lipoprotein hydroxyperoxide by copper catalyzed autoxidation using a published dye based color assay (FOX Assay, see Zadeh, “Methods in Enzymology”, 300, 58 (1999)). Samples containing only LDL and copper sulfate without test materials, serve as a positive control for comparison with identical mixtures containing test materials.

Human Low Density Lipoprotein (Sigma Chemical Company L2139) in phosphate buffered saline pH-7.4 is mixed with copper sulfate. Incubation with effective amounts of compounds of the invention at 25° C. or 37° C. open to air effects oxidation, and the mixture is sampled at time zero and between 3 and 20 hours of incubation for measurement of hydroperoxide in the FOX assay. Samples are read in a microtitre plate reader. Decreased hydroperoxide as measured by the FOX assay reveals the anti-oxidant activity of compounds of the invention and their usefulness for the treatment or prevention of disorders, diseases or conditions associated with oxidation or benefiting from the administration of anti-oxidants. An example of such a condition that would benefit from the treatment of anti-oxidants is cardiovascular disease.

### Example 72

### Measurement of Antioxidant Activity by LDL Oxidation Assay

The method of Esterbauer (Esterbauer, H., Striegl, G., Puhl, H., Rotheneder, M., “Continuous monitoring of in vitro oxidation of human low density lipoprotein”, Free Radic. Res. Commun, 1989; 6(1): 67-75) may be used, with some modification as follows:

The compound is dissolved with an appropriate solubilizing agent in a phosphate buffer solution (PBS, 0.15 M NaCl-0.05 M Na Phosphate Buffer-pH 7.4). The exact concentration is noted (approximately 30-60 mu.g/mL of extract to be measured). To 100 mu.L of this solution is added to 900 mu.L of an oxidizing buffer (made from human IDL (120 mu.L of 5 mg/mL solution with d=1.019-1.063 g/mL, purchased from PerImmune, Rockville, Md.) and copper sulfate (20 mu.L of 10 mM aqueous solution) in 8 mL PBS). A blank sample made with 100 mu.L PBS and 900 mu.L oxidizing buffer is also prepared. Each solution is then transferred to a 1 cm quartz cuvette, and the cuvette is placed into thermostat (37 degrees C.). An HP-8452A Diode Array Spectrophotometer measures optical density at 234 nm (OD sub 234), making a measurement every 5 minutes. The lag time for oxidation is calculated as the maximum of the first derivative of the optical density curve. A standard containing ascorbic acid is run with each assay.

### Example 73

### Measurement and Comparison of HDL, LDL, VLDL and Triglyceride Levels

Compounds or the dosing vehicle alone are administered daily to chow fed male Sprague-Dawley rats or female obese Zucker rats for seven days in the morning by oral gavage in 1.5% carboxymethylcellulose/0.2% Tween-20 (dosing vehicle). Animals are weighed daily and allowed free access to rodent chow and water throughout the study. Orbital blood samples are obtained following a six-hour fast prior to the initial dose and also following the seventh dose. After the seventh dose, animals are sacrificed in the evening and blood serum is assayed for total cholesterol and triglycerides, lipoprotein cholesterol profiles, VLDL plus LDL cholesterol combined (also referred to as apo B containing lipoprotein cholesterol or non-HDL cholesterol), HDL cholesterol, and the ratio of HDL cholesterol to that of VLDL plus LDL cholesterol.

### Example 74

### Measurement and Comparison of HDL, LDL, VLDL and Triglyceride Levels in Humans in Response to Administration of the Compounds

Compounds of the invention are administered daily to human subjects. Other dietary uptake is monitored and held constant between individuals. Blood samples are taken on the day 0, prior to commencing the administration of the compounds, and once weekly for 3 to 6 months. Blood serum is assayed for total cholesterol and triglycerides, lipoprotein cholesterol profiles, VLDL plus LDL cholesterol combined (also referred to as apo B containing lipoprotein cholesterol or non-HDL cholesterol), HDL cholesterol, HDL.sub.2 and HDL.sub.3 cholesterol fractions, and the ratio of HDL cholesterol to that of VLDL plus LDL cholesterol, utilizing standard, commercially available cholesterol tests, such as the VAP test (Atherotech Inc, Birmingham, Ala.) which can reproducibly measure these parameters from a small sample of human blood. Alternatively, HDL.sub.2 and HDL.sub.3 can be measured from blood by the method of Kulkarni (Kulkarni et al. 1997. J. Lipid Res. 38:2353-64) or by the method of Gidez (Gidez et al. 1982. J. Lipid Res. 23:1206-23). Compounds of the invention which increase total HDL, increase HDL.sub.2, decrease total LDL, decrease VLDL, decrease triglyceride, or increase the HDL/total cholesterol or HDL/LDL ratios as determined in such a blood test are useful for the treatment of cholesterol or lipid associated disorders.

### Example 75

### Measurement of Atherosclerotic Lesion Size Using Proteoglycan-Binding-Defective LDL

A nucleic acid construct may be used to generate mice expressing a proteoglycan-binding-defective LDL. The transgenic mice are fed a diet containing 1.2% cholesterol, 0.5% bile salts, and 20% fat for 17 weeks. The mice are then sacrificed, and the aortas are perfusion fixed and analyzed with the en face procedure, in which the entire aorta is pinned out flat, stained with Sudan IV, and analyzed with a morphometric image-analysis system (Image-1/AT) to quantitate the extent of atherosclerosis.

### Example 76

### Measurement of Reduced Hypertension in Living Animals

A pressure transducer is connected to the right carotid artery via a catheter containing heparinized saline. The mean arterial pressure and heart rate are recorded. The rats are anesthetized with nembutal at an initial dose of 35 mg/kg body weight with additional smaller injections as necessary. The compounds are dissolved in a pharmaceutical carrier (such as Abbott's 5% dextrose USP) and injected into the rats via a catheter in the right femoral vein. Positive controls that may be employed include sodium nitroprusside and NaNO2, while NaNO.SUB.3 may be employed as a negative control. The results will show that the compounds provided for in the invention are potent anti-hypertensives, that decreases blood pressure significantly. The peak value of the blood pressure decrease should take a short time to reach, for example approximately one minute, after injection and the blood pressure should start to rise again soon thereafter and should have totally recovered within about approximately 10 to 15 minutes.

### Example 77

### Measurement of the Reduction of Degree of Restenosis After Arterial Injury in High Cholesteric Rabbits

The procedure of Tomaru, as described in U.S. Pat. No. 5,595,974 and further described by Goodman in U.S. Pat. No. 6,022,901 may be used to evaluate the utility of the compounds of the invention to preventing restenosis in high cholesteric rabbits.

### Example 78

### Use in Preventing Restenosis in Humans

The procedure of Tardif et al. (1997), New England J. Med. 337(6):365-67 may be carried out as described by Goodman in U.S. Pat. No. 6,022,901, except that our compounds are examined in place of trans-resveratrol.

### Example 79

### Measurement of Platelet Anti-Aggregating Activity

Platelet anti-aggregating activity may be evaluated in vitro on human platelets stimulated by thrombin in accordance with the method described by Bertele et al. (Science 220, 517, 1983).

### Example 80

### Measurement of the Influence on ADP-Induced Aggregation of Platelets in Rabbits

Aggregation of platelet testing: Rabbit blood is sampled by cardiac puncture from rabbit with silicon-coated syringe. The blood is mixed with 3.8% sodium citrate at 9:1 and spun at 1,000 rpm for 6 minutes. 1 ml of the platelet-rich plasma is transferred to a silicon--coated 2 ml cell, mixed and read for transmittance (Ti), with a spectrophotometer. 0.02 ml of ADP (10 mu.M) is added, stirred, and read for transmittance of the platelet-containing-plasma once per minute and the maximal transmittance (Tm) is obtained within 10 minutes. Spin the blood sample at 3000 rpm for 45 minutes and read for transmittance.

### Example 81

### Measurement of the Effect on Collagen Induced Thrombocytopenia in vivo

Male rats (Charles River, CRL:CD(SD), 400-450 g) are anesthetized with Na pentabarbital (65 mg/kg, Vet Labs, Limited, Inc., Lenexa, Kans.). Two incisions are made to expose both jugular veins. Using an infusion pump (Harvard Apparatus, South Natick, Mass.) and a 5 cc syringe with a 19 g. butterfly, the test compound or vehicle is infused into the left jugular vein at a rate of 0.39 ml/min for 3 min. After 2 min of compound/vehicle infusion, collagen (60 mu.g/kg) (Helena Laboratories, Beaumont, Tex.) is injected with a 1 ml syringe into the right jugular vein. The body cavity is opened and the vena cava is exposed for blood sampling. One min after the collagen injection, compound infusion is stopped and blood is sampled from the vena cava (within 30 sec) with a 3 cc syringe containing 0.3 mg of 4.5% EDTA/Tris (0.1M) (pH 7.35) plus 150 mu.M indomethacin. Platelet rich plasma (PRP) is prepared by centrifuging the blood at 126.times. g for 10 min. Five mu.l of PRP is counted in 20 ml of Isoton.RTM. III in a Coulter Counter. Percent inhibition of collagen induced aggregation is calculated by comparison of the number of platelets counted in treated animals with numbers for animals receiving no collagen and with counts from animals receiving vehicle and collagen. Estimation of potency is based on inhibition of collagen-induced thrombocytopenia.

### Example 82

### Measurement of the in vivo Anti-Psoriatic Effectiveness

A topical formulation comprising a compound of the invention is administered to the affected area of human patients suffering from psoriasis. A control formulation, containing none of the compound of the invention, is applied to a comparable area of the patient. The effectiveness of the compound is determined by analyzing the improvement in inflammation and decrease in proliferative cells at the site at which the compound is applied compared to the site at which control formulation is applied at 3 and 7 days following administration.

### Example 83

### Measurement of Protein Kinase Inhibition

A compound of the invention is mixed with radio-labeled ATP, an appropriate protein kinase and an appropriate substrate in an appropriate buffer. Following incubation the reaction is stopped by spotting onto filter paper and a scintillation counter employed to quantify the difference in ATP addition to the substrate, which measures the amount of protein kinase inhibition, when compared to control.

### Example 84

### Measurement of Inhibition of Neutrophil Activation

A compound of the invention is tested using the protocol of Tudan (Tudan. 1999. Biochem. Pharmacol. 58:1869-80. This test demonstrates the ability of the test compound to inhibit the activation of neutrophils caused by crystals and by chemoattractants such as fMLP.

### Example 85

### Measurement of Inhibition of TPA-Induced Inflammation

A compound of the invention is tested by a modified method of Marks (Marks et al. 1976. Cancer Res. 36:2636) to demonstrate the compound's effectiveness against inflammation induced by application of 12-O-tetradecanoylphorbol-13-acetate (TPA). The compound is applied to an ear of a mouse, followed by application of TPA. Four hours later a biopsy punch of the mouse ear is weighed to measure edema, compared to a biopsy punch of the other ear which received no compound.

### Example 86

### Measurement of COX-1 Inhibition

A compound of the invention is tested by the method of Van der Ouderaa (Van der Ouderaa. 1982. Methods Enzymol. 86:60). The reaction is initiated by the addition of arachidonic acid to a mixture containing the test compound in 0.1 M sodium phosphate (pH 7.4), 1.0 mM phenol, 0.01 mM hemin, and COX-1 enzyme.

### Example 87

### Measurement of the Inhibition of Carrageenan-Induced Inflammation

A compound of the invention is tested by the method of Slowing (Slowing et al. 1994. J. WrhnophEMxol. 43:9) in Wistar rats. Animals receive intradermal injections of Freund's adjuvant into the tail. Seven days later, the test compound is administered, followed one hour later by a suspension of carrageenan in saline solution into the left hind paw. Paw volume is measured by water plethysmography and compared to control.

### Example 88

### Measurement of the Cancer Chemopreventative Activity

C3H/10T1/2 clone 8 cells (ATCC) are treated with a compound of the invention by the method of Mondal (Mondal et al. 1976. Cancer Res. 36:2254-2260). The cells in culture are treated with 3-methylcholanthrene for 24 hours, followed by washing a five days of incubation in fresh medium. TPA is subsequently added to the medium, with or without the test compound. Seven weeks after confluency is reached, fixation with methanol and staining with Giemsa reveals Type II and III transformed foci, which are scored to demonstrate effectiveness of inhibition of two-stage transformation by the test compound.

### Example 89

### Method for Synthesizing Fluoride Derivatives of Compounds of the Invention, Including Stilbenes, Polyphenols and Flavonoids Prior to the Replacement of a Hydroxyl Group or Groups with a Nitrooxy Group or Groups

As it may be desirable to replace one or more hydroxyl groups of a compound of the invention with a fluoride to improve the usefulness of the compound as a therapeutic drug, an example is here provided which describes how to substitute a fluoride for a hydroxyl group that is attached to an aromatic ring, based upon the method of Cramer and Coffman (Cramer and Coffman, 1961 J Org. Chem. 26:4164). Such a procedure will be readily useful without undue experimentation by one of skill in the art for replacing any hydroxyl group with a fluoride for any of the compounds of the invention. As the conditions for fluoridation described in this example are somewhat harsh, for some of the compounds of the invention yields may be improved by building the compound from building blocks rather than fluoridating. When a compound is to have a fluoride in place of a hydroxyl group, as well as to have one or more nitrooxy groups substituted in place of other hydroxyl groups (for example, as in Examples 1 through 59), the fluoride addition reaction should be accomplished first, and the nitrooxy addition reaction performed second.

The following reaction describes the synthesis of fluoride derivatives of resveratrol.

A stainless steel lined autoclave of 400 mL capacity is charged with 250 millimoles of 5-[(E)-2-(4-hydroxy-phenyl)-vinyl]-benzene-1,3-diol (synonym: resveratrol) and evacuated. 500 millimoles of sulfur oxytetrafluoride is introduced, and the reaction mixture is shaken and heated at 150° C. for 9 hours. The gaseous product, principally sulfuryl fluoride, is distilled at −49° C. to −44° C. The remainder is washed with aqueous 5% sodium hydroxide and with water. Upon distillation this liquid will be found to contain a mixture containing the fully and partially fluoridated products, 3-fluoro-5-[(E)-2-(4-hydroxy-phenyl)-vinyl]-phenol, 5-[(E)-2-(4-fluoro-phenyl)-vinyl]-benzene-1,3-diol, 4-[(E)-2-(3,5-difluoro-phenyl)-vinyl]-phenol, and 1,3-difluoro-5-[(E)-2-(4-fluoro-phenyl)-vinyl-benzene. The various products are purified and isolated by chromatography on silica gel.

Following the isolation of a fluoride derivative of resveratrol, the compound may be further modified to contain a nitrooxy group, as described in Examples 1 through 59. This method works without undue experimentation for the addition of fluorides to any of the compounds of the invention.

### Example 90

### Method for the Synthesis of Polyphenols Comprising Two Aromatic Rings Connected by a Linking Group Comprising —(CO)NH—

Polyphenol compounds contemplated in the invention include compounds comprising two aromatic rings connected to one another by a linking group, wherein said linking group comprises the group:

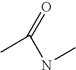

Polyphenol compounds of the following general formula are easily synthesized by this reaction, from readily available starting reagents. 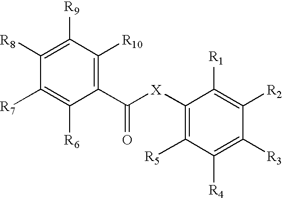
wherein X is NH
and R1-10 are each independently chosen from H or OH

These compounds of the invention are useful as intermediary compounds from which may be subsequently synthesized nitrooxy derivatives as described in Examples 1-59, as well as nitrooxy derivates that may be additionally modified to comprise phosphate, fluoride, ester groups, and other modifications. An example intermediary compound, N-(3,5-dihydroxy-phenyl)-4-hydroxy-benzamide, which is useful in the subsequent preparation of a nitrooxy derivative thereof, is synthesized by the following method.

To a solution of 4-hydroxy-benzoic acid (6 mmol) in dry DMF (15 ml) is added EDCl (9 mmol), HOBt (9 mmol) and triethylamine (12 mmol). After stirring at room temperature for 24 hours, 5-amino-benzene-1,3-diol is added dropwise and the reaction allowed to continue for 48 hours at room temperature under argon. Water (300 ml) is then added and the mixture stirred for 5 min. The product is then extracted with ethyl acetate (5*50ml). The combined organic extracts are washed with brine (40 ml), dried over sodium sulfate, filtered, and the solvent removed. Purification of the product, N-(3,5-dihydroxy-phenyl)-4-hydroxy-benzamide, is achieved by chromatography on silica gel.

Alternatively, the reaction is performed with 5-aminomethyl-benzene-1,3-diol employed in place of 5-amino-benzene-1,3-diol, resulting in the synthesis of N-(3,5-dihydroxy-benzyl)-4-hydroxy-benzamide. This synthesis demonstrates the method for the synthesis of compounds wherein X is NHCH.sub.2 for the general formula of this example. Similarly, as demonstrated, modification to the alkyl group of the phenol will result in the same modification to the linker of the resulting product.

Substitution of the R group connected to the amino reagent provided for in this synthesis description (i.e. substitution of the benzene 1,3-diol group of 5-amino-benzene-1,3-diol), by for example fluorinated, brominated, chlorinated, or acetylated aryl groups, or by heteroaromatic aryl groups, or by C1-18 alkyl groups, or by bicyclic aryl groups, or the like, will result in appropriately modified products, as is obvious to one of skill in the art.

Products synthesized by this method may be advantageously employed as intermediary compounds useful for the synthesis of NO-donating, nitrooxy derivative compounds of the invention.

### Example 91

### Method for the Synthesis of Polyphenols Comprising Two Aromatic Rings Connected by a Linking Group Comprising —C—NH—

Polyphenol compounds contemplated in the invention include compounds comprising two aromatic rings connected to one another by a linking group, wherein said linking group comprises a carbon atom single bonded to a nitrogen atom. Polyphenol compounds of the following general formula are easily synthesized by this reaction, from readily available starting reagents. 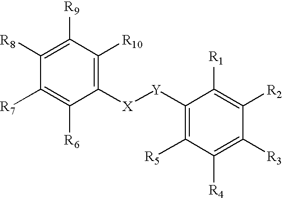
wherein X is CH.sub.2 and Y is NH or, X is NH and Y is CH.sub.2
and R1-10 are each independently chosen from H or OH

These compounds of the invention, are useful as intermediary compounds from which may be subsequently synthesized nitrooxy derivatives as described in Examples 1-59, as well as nitrooxy derivates that may be additionally modified to comprise phosphate, fluoride, ester groups, and other modifications. An example intermediary compound, 5-(4-hydroxy-benzylamino)-benzene 1,3-diol, which is useful in the preparation of a nitrooxy derivative thereof, is synthesized by the following method.

5-amino-benzene-1,3-diol (1.5 mmol) is added to 4-hydroxy-benzaldehyde (1.5 mmol) in benzene (40 ml) and the mixture is heated to reflux under argon for 24 hours using a Dean-Stark trap. The reaction mixture is then concentrated to remove the benzene completely, and the residue is redissolved in methanol (15 ml). While stirring, sodium cyanoborohydride (3 mmol) is added in three portions during 30 min and the reaction mixture is stirred at room temperature for an additional 1 hour. To the reaction mixture is then added a saturated solution of NaCl (100 ml) containing 37% HCl. The reaction mixture is extracted with ethyl acetate (3*50ml). The combined organic layers are washed with brine (10 ml), dried over sodium sulfate and concentrated to furnish the crude product, 5-(4-hydroxy-benzylamino)-benzene 1,3-diol, which is further purified by chromatography on silica gel.

Alternatively, the reaction is performed with 4-hydroxy-phenyl-acetaldehyde employed in place of 4-hydroxy-benzaldehyde, resulting in the synthesis of 5-[2-(4-hydroxy-phenyl)-ethylamino]-benzene-1,3-diol. This synthesis demonstrates the method for the synthesis of compounds wherein X is (CH.sub.2).sub.2 and Y is NH for the general formula of this example. Similarly, as demonstrated, modification to the alkyl group of the phenol will result in the same modification to the linker of the resulting product.

Products synthesized by this method may be advantageously employed as intermediary compounds useful for the synthesis of NO-donating, nitrooxy derivative compounds of the invention.

### Example 92

### Method for the Synthesis of Polyphenols Comprising Two Aromatic Rings Connected by a —CO— Linking Group

Polyphenol compounds contemplated in the invention include compounds comprising two aromatic rings connected to one another by a linking group, wherein said linking group comprises a carbon atom single bonded to an oxygen atom. Polyphenol compounds of the following general formula are easily synthesized by this reaction, from readily available starting reagents. 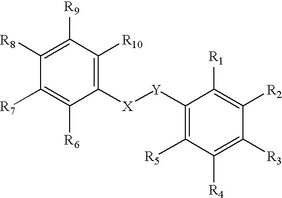
wherein X is CH.sub.2 and Y is oxygen
and R1-10 are each independently chosen from H or OH

These compounds of the invention are useful as intermediary compounds from which may be subsequently synthesized nitrooxy derivatives as described in Examples 1-59, as well as nitrooxy derivates that may be additionally modified to comprise phosphate, fluoride, ester groups, and other modifications. An example intermediary compound, 5-(4-hydroxy-phenoxymethyl)-benzene 1,3-diol, which is useful in the preparation of a nitrooxy derivative thereof, is synthesized by the following method.

Solid tert-butylchlorodimethylsilane (25 mmol) is added to a stirred solution of 4-hydroxy-benzaldehyde (17 mmol) and imidazole (42.5 mmol) in dry N,N-dimethylformamide (100 ml) under argon. After 4 hours, the reaction mixture is poured into water and extracted with ether. The organic extracts are washed with water and brine, dried and concentrated to a colored oil. Filtration through a pad of silica gel with 20% ethyl acetate-hexane as eluent afforded the silyl ether 4-(tert-Butyl-dimethyl-silanyloxy)-benzaldehyde. A solution of 4-(tert-Butyl-dimethyl-silanyloxy)-benzaldehyde (14 mmol) and m-chloroperbenzoic acid (20 mmol) in methylene chloride (100 ml) is heated under reflux for 2 hours and then left overnight at room temperature. The reaction mixture is then extracted into ether followed by washing of the organic layers with aqueous sodium hydroxide (1 M), water and brine, dried and evaporated under reduced pressure to yield a solid. This is preadsorbed on silica gel and then subjected to rapid filtration through a plug of silica gel. A solution of the resulting formate, formic acid 4-(tert-butyl-dimethyl-silanyloxy)-phenyl ester, in methanol (70 ml) is added to potassium carbonate (10 mmol). After 20 minutes, 1-bromomethyl-3,5-bis-(tert-butyl-dimethyl-silanyloxy)-benzene (14 mmol, prepared by essentially the same silanyl protection method as for formic acid 4-(tert-butyl-dimethyl-silanyloxy)-phenyl ester above) is added. After 6 hours the reaction mixture is reduced in volume, water added and the solution acidified with aqueous hydrochloric acid (1 M). It is extracted with ether and the ether extract worked up by the method of Pearson (Pearson et al. 1967 J Org Chem 32:2358). Gradient elution dry column chromatography with 2 to 80% ethyl acetate-hexane as eluents gives 1,3-bis-(tert-butyl-dimethyl-silanyloxy)-5-[4-(tert-butyl-dimethyl-silanyloxy)-phenoxymethyl]-benzene. The 1,3-bis-(tert-butyl-dimethyl-silanyloxy)-5-[4-(tert-butyl-dimethyl-silanyloxy)-phenoxymethyl]-benzene in tetrahydrofuran is treated with tetra-n-butylammonium fluoride trihydrate. After 3.5 hours, water and ether are added. The aqueous layer is acidified with aqueous hydrochloric acid (1 M) and re-extracted with ether. The organic extracts are then worked by the method of Pearson. Filtration through a plug of silica gel (20% ethyl acetate-hexane) produces an oil. The oil is crystallized after trituration with hexane while cooling in an acetone-dry ice bath. Recrystallisation from methylene chloride-hexane affords the product, 5-(4-hydroxy-phenoxymethyl)-benzene 1,3-diol, which is further purified by chromatography on silica gel.

Products synthesized by this method may be advantageously employed as intermediary compounds useful for the synthesis of NO-donating, nitrooxy derivative compounds of the invention.

### Example 93

### Method for the Synthesis of Polyphenols Comprising Two Aromatic Rings Connected by a Linking Group Comprising —C═N—

Polyphenol compounds contemplated in the invention include compounds comprising two aromatic rings connected to one another by a linking group, wherein said linking group comprises a carbon atom double bonded to a nitrogen atom. Polyphenol compounds of the following general formula are easily synthesized by this reaction, from readily available starting reagents. 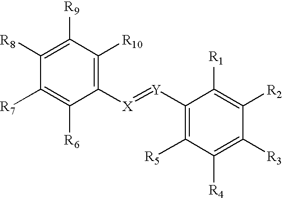
wherein X is CH and Y is N or, X is N and Y is CH
and R1-10 are each independently chosen from H or OH

These compounds of the invention are useful as intermediary compounds from which may be subsequently synthesized nitrooxy derivatives as described in Examples 1-59, as well as nitrooxy derivates that may be additionally modified to comprise phosphate, fluoride, ester groups, and other modifications. An example intermediary compound, 5-{[(E)-4-hydroxy-phenylimino]-methyl}-benzene 1,3-diol, which is useful in the preparation of a nitrooxy derivative thereof, is synthesized by the following method.

A solution of 3,5-dihydroxy-benzaldehyde (1 mmol) and 4-amino-phenol (1 mmol) in toluene (5 ml) is heated to reflux in a Dean and Stark apparatus for 16 hours. After the solvent is removed in vacuo, the product 5-{[(E)-4-hydroxy-phenylimino]-methyl}-benzene 1,3-diol is recrystallized from methanol and further purified by chromatography on silica gel.

Products synthesized by this method may be advantageously employed as intermediary compounds useful for the synthesis of NO-donating, nitrooxy derivative compounds of the invention.

### Example 94

### General Method for the Synthesis of Stilbenes (and Dihydrostilbenes) Comprising Two Aromatic Rings Connected by a Linking Group Comprising —C═C—

Stilbene compounds contemplated in the invention include compounds comprising two aromatic rings connected to one another by a linking group, wherein said linking group comprises a carbon atom double bonded to another carbon atom. Stilbene compounds of the following general formula are easily synthesized by this reaction, from readily available starting reagents. 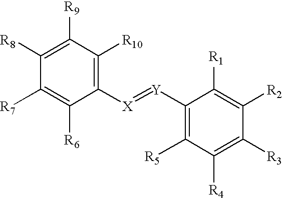
wherein X is CH and Y is CH
and R1-10 are each independently chosen from H or OH

These compounds of the invention are useful as intermediary compounds from which may be subsequently synthesized nitrooxy derivatives as described in Examples 1-59, as well as nitrooxy derivates that may be additionally modified to comprise phosphate, fluoride, ester groups, and other modifications. An example intermediary compound, resveratrol (synonym: 5[(E)-2-(4-hydroxy-phenyl)-viny]-benzene 1,3-diol) which is useful in the preparation of a nitrooxy derivative thereof, is synthesized by the following method.

A mixture of 3,5-dihydroxy-benzyl-bromide (10 mmol) and trimethyl phosphite (30 mmol) in a sealed tube is heated at 180° C. in an oil bath for 8 hours. After the mixture is cooled, the excess trimethyl phosphite is removed in vacuo. Purification of the residue by short flash column chromatography gives the product, (3,5-dihydroxy-benzyl)-phosphonic acid dimethyl ester. To (3,5-dihydroxy-benzyl)-phosphonic acid dimethyl ester in a well-stirred suspension also containing freshly powdered KOH (2 mmol), 18-crown-6 (0.1 mmol) in 2 ml of CH.sub.2Cl.sub.2 is added the aromatic aldehyde 4-hydroxy-benzaldehyde (1 mmol) at room temperature. After the mixture is stirred for 6 hours, the mixture is diluted with 15 ml CH.sub.2Cl.sub.2 and washed with water (10 ml) and brine (2*10 ml). The organic layer is dried over magnesium sulfate and concentrated in vacuo. The reside is dissolved in 2 ml of Ch.sub.2Cl.sub.2. To this solution is added Girard's reagent T (0.5 mmol) and AcOH (5 mmol) and the resulting mixture is stirred for 2 hours at room temperature. The insoluble material is filtered off, the filtrate is concentrated in vacuo, and the residue is dissolved in 15 ml EtOAc.

The solution is washed with brine (3*10 ml) and dried aver magnesium sulfate, and the solvent removed in vacuo to yield resveratrol in a mix of E and Z isomers. To the solution of this mixture in heptane (5 ml) is added a catalytic amount of iodine and then heated to reflux for 12 hours. The reaction mixture is diluted wth 20 ml of ether and washed with saturated aqueous sodium bisulfite (10 ml) and brine (2*10 ml). The organic layer is dried over magnesium sulfate and concentrated in vacuo to provide the desired E-resveratrol.

This method is advantageously employed to synthesize any of the stilbene compounds which are intermediary compounds for the synthesis of NO-donating, nitrooxy derivative compounds contemplated by this invention.

Dihydrostilbenes, which are derivatives of the corresponding stilbenes with the difference of having a single bond between the two carbon atoms of the linking group, may be advantageously synthesized from the stilbene parent compound. The general method is as follows.

A stilbene (1 mmol) in ethanol (120 ml) is hydrogenated at 40 psi in the presence of 10% palladium on charcoal (60 mg) for 18-24 hours. The catalyst is removed by filtration through a Celite pad, and the solvent is evaporated from the filtrate to afford the dihydrostilbene derivative.

This method is advantageously employed to synthesize any of the dihydrostilbene compounds which are intermediary compounds for the synthesis of NO-donating, nitrooxy derivative compounds contemplated by this invention.

### Example 95

### Method for Synthesizing Phosphate-Derivative Compounds of the Invention

It may be advantageous to substitute phosphate groups in place of hydroxyl groups for some compounds of the invention, as phosphate groups can alter the metabolism and the half life of a compound in serum. As an example but not to be limited by this example, the synthesis of phosphate derivatives of resveratrol, which advantageously occurs following the replacement of other hydroxyl groups by fluoride, esters and nitrooxy groups (i.e. nitrate or nitric ester groups), is described herein.

First, a single nitrooxy substituted derivative (e.g. 3-[(E)-2-(4-hydroxy-phenyl)-vinyl]-5-nitrooxy-phenol) of resveratrol is synthesized, isolated and purified as described in Example 1. The single nitrooxy substituted derivative (4 g) and N,N-(dimethylamino)pyridine (0.2 g) in anhydrous acetonitrile (30 ml) is cooled to −10° C., and carbon tetrachloride (5 equiv) and DIEA (2 equiv) is added. The mixture is stirred at −10° C. for 30 min under argon, dibenzyl phosphate (1 equiv) is added, and the solution is stirred for 12 hours and then poured into 0.5 M monobasic potassium phosphate. The mixture is extracted with ethyl acetate, and removal of solvent in vacuo from the organic phase yields a colored oil. This is subjected to flash column chromatography (4:1 hexane/ethyl acetate) and the phosphate ester products are recovered as a colored oil.

To a solution of the phosphate ester products in anhydrous dichloromethane (15 ml) at 0° C. is added bromotrimethylsilane (2 equiv) and the mixture is stirred for 2 hours. Water (10 ml) is added, the solution is stirred for 1 hour and washed with ethyl acetate, and the aqueous phase is freeze-dried to a white solid. To a solution of the solid in ethanol (30 ml) is added sodium methoxide (0.6 g) and the suspension is stirred for 12 hours. Solvent is removed in vacuo, and the resulting colored oil is dissolved in water. The solution is washed with ethyl acetate and then freeze-dried to afford a high yield, high purity colorless solid comprising a mixture of derivatives of resveratrol with phosphate, hydroxyl and nitrooxy groups. The desired derivative(s) are isolated and purified by chromatography on silica gel.

This synthesis process may be advantageously employed to substitute phosphates in place of hydroxyl groups for any of the compounds of the invention.

### Example 96

### Method for Synthesizing Acetyl-Derivative Compounds of the Invention

It may be advantageous to substitute acetyl groups in place of hydroxyl groups for some compounds of the invention, as certain acetyls can alter the degree of lipophilicity and thus modify the rate of metabolism and half life of a compound in the serum. For example, replacing one or more of the hydroxyl groups of resveratrol to form an acetate derivative of resveratrol reduces the rate of metabolism and extends the half life in the serum. The synthesis of acetate derivatives of resveratrol, which advantageously occurs prior to addition of one or more nitrate (i.e. ONO.sub.2, or nitric ester) groups, and prior to addition of phosphate groups if such is desired, but following fluoridation, is described herein.

Resveratrol (0.5 millimoles) is dissolved in dry dichloromethane (5 ml). Dry pyridine in excess is added followed by 1 millimole of acetic anhydride. The resulting solution is stirred at room temperature for 5 hours. The reaction mixture is concentrated and redissolved in dichloromethane (20 ml). The organic layer was washed with a hydrogen chloride solution (0.1 M, 10 ml), sodium bicarbonate (saturated, 10 ml), and brine. The organic layer was dried with magnesium sulfate, filtered and concentrated to give a mixture in high yield and purity of resveratrol acetate derivatives wherein one, two or all three of the hydroxyl groups was replaced by acetate. The various products are purified and isolated by chromatography on silica gel.

The acetate derivatives are also synthesized using an acetyl halide (such as acetyl chloride) or activated acetate (such as the N-hydroxysuccinimide ester). Other esters of compounds of the invention are similarly synthesized using the same procedure, replacing the acetic anhydride with another activated ester or acid halide. Examples of such esters which can be substituted for any hydroxyl group on any of the compounds contemplated by the invention are described by the formula: 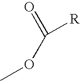
wherein R can be C₁₋₁₈, aryl, heteroaryl, and optionally substituted derivatives thereof.

The nitrating synthesis step, as described in Examples 1 through 59, may be advantageously performed following the synthesis of the acetate derivative and isolation and purification by chromatography on silica gel.

### Example 97

### Method of Synthesizing Methoxy and Ethoxy Derivatives of Compounds of the Invention, Used as Intermediary Compounds from which are Synthesized Nitrooxy Derivatives, which are All Compounds Contemplated by the Invention

It may be advantageous with some compounds of the invention to have methoxy (OCH.sub.3) or ethoxy (OCH.sub.2CH.sub.3) groups present for the R group, as methoxy and ethoxy groups are known to be lipophilic and thus may modify the half life of a drug in vivo without reducing its activity. Numerous methoxy and ethoxy derivatives of aryl hydrocarbons (for example of benzene, phenol and the like) are known and readily available from commercial sources, or easily synthesizable by well known methods. The building blocks for making compounds of the stilbene class, and of other polyphenol classes, are therefore readily available, and may be utilized as in Examples 90 through 94. Polyphenols and stilbenes, as defined in this application, may therefore be synthesized such that the R groups may independently, optionally comprise methoxy (OCH.sub.3) or ethoxy (OCH.sub.2CH.sub.3).

### Example 98

### Method of Demonstrating Anti-Fungal Activity of Compounds of the Invention

Fungicidal compounds of the invention are demonstrated using methods as taught in U.S. Pat. No. 6,165,998. Briefly, exposing about 10.sup.6 C. albicans or S. cerevisiae cells to 25 .mu.g/ml of a fungicidal compound of the invention for 45 minutes leaves no detectable colony forming units.

In addition, fungicidal compounds of the invention are efficacious in a murine model for systemic candidiasis. Fungicidal compounds of the invention prolong mean and median survival times of treated mice. The compounds are administered IP producing a similar survival pattern as that produced by the positive control compound fluconazole administered orally. Both the fungicidal compounds of the invention and fluconazole reduce recoverable colonies from the kidneys of treated animals. Fungicidal compounds of the invention are also efficacious when administered orally to mice with an established systemic Candida infection. The compound given orally is similar in efficacy to fluconazole as measured by survival time, per cent cures and kidney burden. Fungicidal compounds of the invention are also effective against systemic candidiasis caused by a strain of C. albicans resistant to fluconazole.

### Example 99

### Method of Demonstrating Anti-Cancer Activity of Compounds of the Invention

Anti-cancer activity of compounds of the invention is demonstrated, as taught in U.S. Pat. No. 5,145,839, using the following animal model of cancer, and treating with compounds of the invention.

O BALB C mice bearing lymphoma YC8 (ascitic form) and Swiss mice bearing Ehrlich ascitic cells (20-22 grams, Charles River breeding) are distributed at random in sets of 10. Each set receives, respectively:
Set I Control. tumor cells and NaCl isotonic solution (0.2 ml/mouse, twice/day, i.p. route)
Set II: Mice bearing tumor cells receive a compound of the invention 0.2 ml/mouse, twice/day, delivered i.p.
Set III: Mice bearing tumor cells receive a compound of the invention: 0.2 ml/mouse twice/day, i.p. route and a chemotherapeutic agent administered i.p.
Set IV: Mice bearing tumor cells receive a compound of the invention: 0.2 ml/mouse twice/day, administered i.m.

Ascitic tumor cells are taken in sterile medium from mice bearing these cells for 15-20 days. 0.1 ml of ascitic suspension is mixed with 10 ml of buffered solution (pH 7.2): (NaCl 7.2 g/l; Na.sub.2 HPO.sub.4 4.3 g/l and KH.sub.2 PO.sub.4 0.4 g/l). The number of cells is determined (by Malassez cell) and cellular suspension diluted in order to get cell number close to 40.000-50.000/ml. 0.1 ml of this suspension is immediately injected by i.p. route to mice in sets I, II and III and by i.m. route to mice in set IV.

48 hours after injection of tumor cells: the mice of set II receive (i.p.) the compound of the invention, heated at 37.degree. and filtered on millipore, treatment for five consecutive days; the mice of set III are treated (i.p.) by a mixture of the compound of the invention and one of the antibiotics for 5 consecutive days; the mice of set I (control) receive (i.p.) only isotonic solution for 5 consecutive days; the mice of set IV receive (i.m.) the compound of the invention for 15 consecutive days. Mice are observed for one or two months after cessation of treatment. Only survivors in excellent physical condition are taken into consideration. Compounds of the invention are therefore useful as anti-cancer agents, as demonstrated in this test.

### Example 100

### Method of Demonstrating Anti-Diabetic Activity of Compounds of the Invention

The hypoglycemic activity of compounds of the invention is demonstrated using methods taught in U.S. Pat. No. 6,410,596. This test demonstrates the activity of the compounds of the invention in reducing plasma glucose levels in C57BL/ks diabetic (db/db) mice, i.e., an art-recognized model of non-insulin dependent diabetes mellitus (NIDDM).

### Example 101

### Method of demonstrating anti-viral activity of compounds of the invention

In Vivo Evaluation of Robustaflavone in a Murine Influenza Model

In vivo experiments are run to demonstrate that compounds of the invention are efficacious against an experimentally induced influenza virus infection in specific pathogen-free BALB/c mice. These experiments are performed essentially as taught in Example 11 of U.S. Pat. No. 6,399,654 with compounds of the invention substituted in place of Robustaflavone.

1. A stilbene compound comprising the following structure: 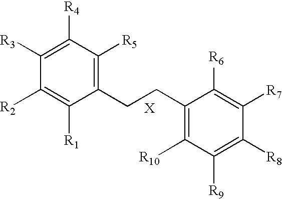
wherein
R1, R2, R3, R4, R5, R6, R7, R8, R9 and R10 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [OCOR11], O-sulfate [the sulfate conjugate], or O-glucoronidate [the glucoronic (AKA glucuronic) acid conjugates], with the proviso that at least one of R1-R10 is nitrooxy; and
wherein OCOR11 means 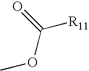
wherein R11 can be C₁₋₁₈, aryl, heteroaryl, and optionally substituted derivatives thereof; and
wherein X can be a single, double or triple bond.

2. A method for treating cardiovascular, cholesterol or lipid related disorders in a patient comprising administering to a patient in need of treatment a therapeutically effective amount of a compound according to claim 1.

3. A method for inducing expression of ApoA1 while providing anti-oxidant activity in a patient comprising administering to said patient the compound of claim 1.

4. A method for reducing serum cholesterol in a patient comprising administering to said patient the compound of claim 1.

5. A flavonoid compound comprising the following structure: 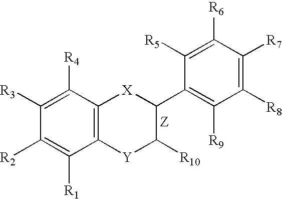
wherein
R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [OCOR11], O-sulfate [the sulfate conjugate], or O-glucoronidate [the glucoronic (AKA glucuronic) acid conjugates], with the proviso that at least one of R1-R10 or R12 or R13 is nitrooxy;
OCOR11 means 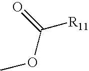
wherein R11 can be C₁₋₁₈, aryl, heteroaryl, and optionally substituted derivatives thereof;
X can be O or CR12;
Y can be CO [a ketone still maintaining the 6 atom ring structure] or CR13; and Z can be a single or a double bond.

6. A method for treating cardiovascular, cholesterol or lipid related disorders in a patient comprising administering to a patient in need of treatment a therapeutically effective amount of a compound according to claim 5.

7. A method for inducing expression of ApoA1 while providing anti-oxidant activity in a patient comprising administering to said patient the compound of claim 5.

8. A method for reducing serum cholesterol in a patient comprising administering to said patient the compound of claim 5.

9. An isoflavonoid compound comprising the following structure: 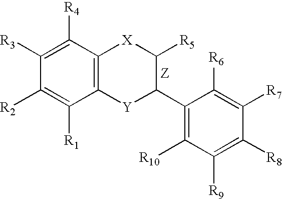
wherein
R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 or R12 or R13 is nitrooxy;
OCOR11 means 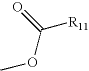
wherein R11 can be C₁₋₁₈, aryl, heteroaryl, and optionally substituted derivatives thereof;
X can be O or CR12;
Y can be CO [a ketone still maintaining the 6 atom ring structure] or CR13; and Z can be a single or a double bond.

10. A method for treating cardiovascular, cholesterol or lipid related disorders in a patient comprising administering to a patient in need of treatment a therapeutically effective amount of a compound according to claim 9.

11. A method for inducing expression of ApoA1 while providing anti-oxidant activity in a patient comprising administering to said patient the compound of claim 9.

12. A method for reducing serum cholesterol in a patient comprising administering to said patient the compound of claim 9.

13. A chalcone compound comprising the following structure: 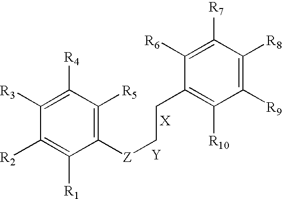
wherein
R1, R2, R3, R4, R5, R6, R7, R8, R9, R10, R12 and R13 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 or R12 or R13 is nitrooxy;
OCOR11 means 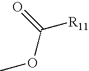
wherein R11 can be C₁₋₁₈, aryl, heteroaryl, and optionally substituted derivatives thereof;
X can be a single or a double bond;
Y can be a single or a double bond; and
Z can be CO [a ketone] or CR13;
with the proviso that X and Y are not both double bonds, and if Z is CO then Y is not a double bond.

14. A method for treating cardiovascular, cholesterol or lipid related disorders in a patient comprising administering to a patient in need of treatment a therapeutically effective amount of a compound according to claim 13.

15. A method for inducing expression of ApoA1 while providing anti-oxidant activity in a patient comprising administering to said patient the compound of claim 13.

16. A method for reducing serum cholesterol in a patient comprising administering to said patient the compound of claim 13.

17. A polyphenol compound comprisng the following structure: 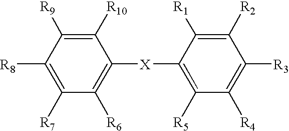
wherein
R1, R2, R3, R4, R5, R6, R7, R8, R9 and R10 may each be independently hydrogen, hydroxyl [OH], nitrooxy [ONO.sub.2], methoxy [OCH.sub.3], ethoxy [OCH.sub2CH.sub.3], fluoride [F], phosphate, acetyl [the ester OCOR11], O-sulfate [the sulfate conjugate], or O-glucoronidate [the glucoronic (AKA glucuronic) acid conjugates], with the proviso that at least one of R1-R10 is nitrooxy;
OCOR11 means 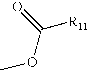
wherein R11 can be C₁₋₁₈, aryl, heteroaryl, and optionally substituted derivatives thereof;
X can be C, S, (CO), SO, AKA ketone, (SO.sub.2)N, (CO)C, (CO)N, (CO)O, C—N [single bond], C═N [double bond], C—O, N—O, N—N [single bond], or N═N [double bond].

18. A method for treating cardiovascular, cholesterol or lipid related disorders in a patient comprising administering to a patient in need of treatment a therapeutically effective amount of a compound according to claim 17.

19. A method for inducing expression of ApoA1 while providing anti-oxidant activity in a patient comprising administering to said patient the compound of claim 17.

20. A method for reducing serum cholesterol in a patient comprising administering to said patient the compound of claim 17.
